SEQUENCE LISTING (122 sequences)

SEQ ID NO: 1 - protein, 375 aa, Gallus gallus
MQKLAVYVYIYLFMQIAVDPVALDGSSQPTENAEKDGLCNACTWRQNTKSSRIEAIKIQILSKLRLEQAPNISRDVIKQLLPKAPPLQELIDQYDVQRDDSSDGSLEDDDYHATTETIITMPTESDFLVQMEGKPKCCFFKFSSKIQYNKVVKAQLWIYLRQVQKPTTVFVQILRLIKPMKDGTRYTGIRSLKLDMNPGTGIWQSIDVKTVLQNWLKQPESNLGIEIKAFDETGRDLAVTFPGPGEDGLNPFLEVRVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 2 - DNA, 1128 bp, Gallus gallus
atgcaaaagctagcagtctatgtttatatttacctgttcatgcagatcgcggttgatccagtggctctggatggcagtagtcagcccacagagaacgctgaaaaagacggactgtgcaatgcttgtacgtggagacagaatacaaaatcctccagaatagaagccataaaaattcaaatcctcagcaaactgcgcctggaacaagcacctaacattagcagggacgttattaagcagcttttacccaaagctcctccactgcaggaactgattgatcagtatgatgtccagagggacgacagtagcgatggctctttggaagacgatgactatcatgccacaaccgagacgattatcacaatgcctacggagtctgattttcttgtacaaatggagggaaaaccaaaatgttgcttctttaagtttagctctaaaatacaatataacaaagtagtaaaggcacaattatggatatacttgaggcaagtccaaaaacctacaacggtgtttgtgcagatcctgagactcattaagcccatgaaagacggtacaagatatactggaattcgatctttgaaacttgacatgaacccaggcactggtatctggcagagtattgatgtgaagacagtgctgcaaaattggctcaaacagcctgaatccaatttaggcatcgaaataaaagcttttgatgagactggacgagatcttgctgtcacattcccaggaccgggtgaagatggattgaacccatttttagaggtcagagttacagacacaccgaaacggtcccgcagagattttggccttgactgtgatgagcactcaacggaatcccgatgttgtcgctacccgctgacagtggatttcgaagcttttggatgggactggattatagcacctaaaagatacaaagccaattactgctccggagaatgtgaatttgtgtttctacagaaatacccgcacactcacctggtacaccaagcaaatcccagaggctcagcaggcccttgctgcacacccaccaagatgtcccctataaacatgctgtatttcaatggaaaagaacaaataatatatggaaagataccagccatggttgtagatcgttgcgggtgctcatga

SEQ ID NO: 3 - protein, 311 aa, Gallus gallus
PAAGKKLPRLPKCARCRNHGYSSPLKGHKRFCMWRDCQCKKCSLIAERQRVMAVQVALRRQQAQEEELGISHPVPLPSAPEPVVKKSSSSSSCLLQDSSSPAHSTSTVAAAAASAPPEGRMLIQDIPSIPSRGHLESTSDLVVDSTYYSSFYQPSLYPYYNNLYNYSQYQMAVATESSSSETGGTFVGSAMKNSLRSLPATYMSSQSGKQWQMKGMENRHAMSSQYRMCSYYPPTSYLGQGVGSPTCVTQILASEDTPSYSESKARVFSPPSSQDSGLGCLSSSESTKGDLECEPHQEPGAFAVSPVLEGE

SEQ ID NO: 4 - DNA, 1493 bp, Gallus gallus
ccggcggcgggcaagaagctgccgcgtctgcccaagtgtgcccgctgccgcaaccacggctactcctcgccgctgaaggggcacaagcggttctgcatgtggcgggactgccagtgcaagaagtgcagcctgatcgccgagcggcagcgggtgatggccgtgcaggttgcactgaggaggcagcaagcccaggaagaggagctggggatcagccaccctgtacccctgcccagtgcccctgagccagttgtcaagaagagcagcagcagcagctcctgtctcctgcaggacagcagcagccctgctcactccacgagcacggtggcagcagcagcagcgagcgcaccaccagagggacggatgctcattcaggacatcccttccatccccagcagagggcacttggagagcacgtctgatttggttgtggactccacctactacagcagtttttaccagccatccctgtatccttactataacaacctgtacaactactcccagtaccaaatggcagtggccactgagtcttcctcaagtgagacagggggtacgtttgtagggtcagccatgaaaaacagccttcgaagcctcccagcaacatacatgtcaagccagtcaggaaaacagtggcagatgaagggaatggagaaccgccatgccatgagctcccagtaccggatgtgctcctactacccgcccacctcatacctgggccagggggttggcagtcccacctgcgtcacacagatactggcctcggaggacaccccctcctactcagagtcgaaagcgagagtgttttcgccgcccagcagccaggactcgggcctggggtgcctgtcgagcagcgagagcaccaagggagacctggagtgcgagccccaccaagagcccggcgccttcgcggtgagcccggttcttgagggcgagtaggcgcggcgtcgggcggctgctgcgcggcgttcactgttgccttgttctgttggggttgcgggggggcgttgggtttcttctttccggggcggggggggcacggcggggccgcggccgggccggcggggcggggcggggcgggacggggcggggcggagccgcgcgggggccgcagtccgggccggggccgccgtcgggtctcggcccgctcccgtcggggcggagcgtccgacgatcggcctccacgaaacgcggtgccgtgatgtgtttgtagtggttcctcgtaggctccagacgttttctcctcgtatcgccaaattaacgcgttttgcatattacagttgagtgcctcgacttagattgcaatataagcggccagcaaacaagtctcaaaaaaaagttacgtgcgtttctgcgagtgttattttgttaagaacggctcacagtgtcctcttcctgtgttacagaagccaacctgaaatgaaactagtctggaaaaattcattgttctctgtagttgcagctgtacctgaaataaaaatgttattgatgactga

SEQ ID NO: 5 - protein, 130 aa, Gallus gallus
MAGGIVRSPAAWRGGAALLGKVARQEFSANVIREEEPLWTRSALRSMIFHRKLLRFFLAAPQKAVVGLSGAEDCGAPLLGRLMIVGEKCAASLGLTDGFRMAVRYPPSVPSDYRARLCILGGRQLGQPPG

SEQ ID NO: 6 - DNA, 393 bp, Gallus gallus
atggccggcgggatcgttaggtcgccggccgcctggcgcggtggcgccgctctcttgggaaaagtcgcccgccaggagttctccgccaacgttatccgcgaggaggagccgttgtggacgaggagtgccttgcgttccatgatatttcaccgcaagctcctacgctttttcctagccgctccccagaaggccgttgtcgggttatctggagcagaagattgtggcgcacctcttcttgggcgtttgatgattgttggcgagaagtgtgctgctagcctgggcttgaccgatggattccggatggctgtgagatacccaccctcagtcccttcagactaccgcgcgcggctctgtattctgggtggccgtcagttgggccagcctcctggctaa

SEQ ID NO: 7 - DNA, 325 bp, Gallus gallus
cgaagaaccgagcgctgctggccttaaagtcccaccaaaactctgaagaaacgaagccagacccggcactcagcgggcagcccgcgcctcccgccgccccacagtgccgcgcgcgtgcatttgcatagcgcggtgctcgcagggggaaactcaccccctcaagtccgccccccgcttcccgcccgctgtcccgcacctcatcagtgctgtgcgctgtctgtgtcccccagcacgcactctttgctgttcttacccggaggcttgccctatccttgaggtttctattttttaggctataaataccgcctaggaggtagagatattc

SEQ ID NO: 8 - DNA, 394 bp, Gallus gallus
cagacagacgtcaggctttctaagcctggactgagtaagagcggaagagctccacagcactctgagtgcgcacagaccgcgcgtacagcgcacagccgcgcggccgctccttcaggcactgccgacgacagcccaggcggaggtcctgagcgccggcgctaaatttgcataaagaactacccaggagccctcgcgcgcggaaacgggcaaaaaggggcttctaatatggaaatattacgccgaatcgcgttacaaatcggctaagcgggcctaagagttaacaagatgtgctattaagcggagccttttggtgggaagaaatggagtagtcactgtgttctaaaagaacttgcagaatgagcctttaaataccgcagtctcgatgctcttagtc

SEQ ID NO: 9 - DNA, 287 bp, Gallus gallus
tgaattgtgggacggcggaagacgggctcccgccccgcccctatatgcaaagcagagaacttcccgccgtgcaccgcgcgcaatcggaagagaatttgggcacttcagacccaaaaaaaaacccaaaacttctgcgaaaaagaaagaatctcagcggagtaaatagggatttttgttaaagaggtgataccagaagaagaaatatgcaaatacaacgccagctcaccgctacttaaaaatcatgatataatagaggcttaaatactgtccgagagacactggggttt

SEQ ID NO: 10 - DNA, 783 bp, Gallus gallus
gtccagccatccacctcccaccaatacttccccactgaaccatgtccctcagtagcacagggtttgtggaacgcctcctgggacggtgcctcccccacctgccacgcagcccattccagcacctgacacttctggagacgaaatttttcctaacgtccaacctgagtctcccctggtgcaacttgaggctgttcccctgactcccatcgctagttacgtgggaagaaaagacccctaagaccaccccgtgcaaccaccagcccatccccaccacgcccactgaccgggcccctcagtgccacagcagcacggttctcgagcgcttcgcaggacggtgagcactgcccggaacctctgcacggcctcagcaacgcgactttcagccggggtcgctgcccaggagccggcggcttcggagcgcagagcgaggcgggagagctccggccgcgggaggctcagtgtcacgcagagcgcgggacgagcgctccgagccctcccagtgccgcccccaaggcagggcggccggcgcagctccccgcagcccgccagtgggaaggctctgctttgcataacgcgcaaggcctgctgggaggaaagcggagcgagaaagagcgttaacgtgcgccgagtgttttagagcaaaagcattcagacctgaagcagcgctgagagatgcctctgccgcccatttactggaaacgttcagacccaccgcaagtcaccgtgaccttgagacactgagctgttggccgttatatagcacttggggcagctcgtagcttt

SEQ ID NO: 11 - RNA, 19 nt, Artificial
  RNA for silencing
ccaguugucaagaagagca

SEQ ID NO: 12 - RNA, 19 nt, Artificial
  RNA for silencing
ggaugcucauucaggacau

SEQ ID NO: 13 - RNA, 19 nt, Artificial
  RNA for silencing
cccuguauccuuacuauaa

SEQ ID NO: 14 - RNA, 19 nt, Artificial
  RNA for silencing
gccacugagucuuccucaa

SEQ ID NO: 15 - RNA, 19 nt, Artificial
  RNA for silencing
ccagcaacauacaugucaa

SEQ ID NO: 16 - RNA, 19 nt, Artificial
  RNA for silencing
ccugcgucacacagauacu

SEQ ID NO: 17 - RNA, 19 nt, Artificial
  RNA for silencing
ggaguaguuguacagguug

SEQ ID NO: 18 - RNA, 19 nt, Artificial
  RNA for silencing
gacuggcuugacauguaug

SEQ ID NO: 19 - RNA, 19 nt, Artificial
  RNA for silencing
auggcgguucuccaucccu

SEQ ID NO: 20 - RNA, 19 nt, Artificial
  RNA for silencing
aagcuagcagucuauguuu

SEQ ID NO: 21 - RNA, 19 nt, Artificial
  RNA for silencing
gcuagcagucuauguuuau

SEQ ID NO: 22 - RNA, 19 nt, Artificial
  RNA for silencing
cgcugaaaaagacggacug

SEQ ID NO: 23 - RNA, 19 nt, Artificial
  RNA for silencing
aaagacggacugugcaaug

SEQ ID NO: 24 - RNA, 19 nt, Artificial
  RNA for silencing
agacggacugugcaaugcu

SEQ ID NO: 25 - RNA, 19 nt, Artificial
  RNA for silencing
ugcuuguacguggagacag

SEQ ID NO: 26 - RNA, 19 nt, Artificial
  RNA for silencing
uacaaaauccuccagaaua

SEQ ID NO: 27 - RNA, 19 nt, Artificial
  RNA for silencing
aauccuccagaauagaagc

SEQ ID NO: 28 - RNA, 19 nt, Artificial
  RNA for silencing
uccuccagaauagaagcca

SEQ ID NO: 29 - RNA, 19 nt, Artificial
  RNA for silencing
uagaagccauaaaaauuca

SEQ ID NO: 30 - RNA, 19 nt, Artificial
  RNA for silencing
gccauaaaaauucaaaucc

SEQ ID NO: 31 - RNA, 19 nt, Artificial
  RNA for silencing
aaauucaaauccucagcaa

SEQ ID NO: 32 - RNA, 19 nt, Artificial
  RNA for silencing
auucaaauccucagcaaac

SEQ ID NO: 33 - RNA, 19 nt, Artificial
  RNA for silencing
auccucagcaaacugcgcc

SEQ ID NO: 34 - RNA, 19 nt, Artificial
  RNA for silencing
acugcgccuggaacaagca

SEQ ID NO: 35 - RNA, 19 nt, Artificial
  RNA for silencing
caagcaccuaacauuagca

SEQ ID NO: 36 - RNA, 19 nt, Artificial
  RNA for silencing
gcaccuaacauuagcaggg

SEQ ID NO: 37 - RNA, 19 nt, Artificial
  RNA for silencing
cauuagcagggacguuauu

SEQ ID NO: 38 - RNA, 19 nt, Artificial
  RNA for silencing
gcagcuuuuacccaaagcu

SEQ ID NO: 39 - RNA, 19 nt, Artificial
  RNA for silencing
uuccugcaguggaggagcu

SEQ ID NO: 40 - RNA, 19 nt, Artificial
  RNA for silencing
cugauugaucaguaugaug

SEQ ID NO: 41 - RNA, 19 nt, Artificial
  RNA for silencing
gacgaugacuaucaugcca

SEQ ID NO: 42 - RNA, 19 nt, Artificial
  RNA for silencing
ccgagacgauuaucacaau

SEQ ID NO: 43 - RNA, 19 nt, Artificial
  RNA for silencing
ugccuacggagucugauuu

SEQ ID NO: 44 - RNA, 19 nt, Artificial
  RNA for silencing
auggagggaaaaccaaaau

SEQ ID NO: 45 - RNA, 19 nt, Artificial
  RNA for silencing
aaccaaaauguugcuucuu

SEQ ID NO: 46 - RNA, 19 nt, Artificial
  RNA for silencing
ccaaaauguugcuucuuua

SEQ ID NO: 47 - RNA, 19 nt, Artificial
  RNA for silencing
aauguugcuucuuuaaguu

SEQ ID NO: 48 - RNA, 19 nt, Artificial
  RNA for silencing
uguugcuucuuuaaguuua

SEQ ID NO: 49 - RNA, 19 nt, Artificial
  RNA for silencing
guuuagcucuaaaauacaa

SEQ ID NO: 50 - RNA, 19 nt, Artificial
  RNA for silencing
aauacaauauaacaaagua

SEQ ID NO: 51 - RNA, 19 nt, Artificial
  RNA for silencing
uacaauauaacaaaguagu

SEQ ID NO: 52 - RNA, 19 nt, Artificial
  RNA for silencing
uauaacaaaguaguaaagg

SEQ ID NO: 53 - RNA, 19 nt, Artificial
  RNA for silencing
caaaguaguaaaggcacaa

SEQ ID NO: 54 - RNA, 19 nt, Artificial
  RNA for silencing
aguaguaaaggcacaauua

SEQ ID NO: 55 - RNA, 19 nt, Artificial
  RNA for silencing
aggcacaauuauggauaua

SEQ ID NO: 56 - RNA, 19 nt, Artificial
  RNA for silencing
uuauggauauacuugaggc

SEQ ID NO: 57 - RNA, 19 nt, Artificial
  RNA for silencing
guccaaaaaccuacaacgg

SEQ ID NO: 58 - RNA, 19 nt, Artificial
  RNA for silencing
aaaccuacaacgguguuug

SEQ ID NO: 59 - RNA, 19 nt, Artificial
  RNA for silencing
accuacaacgguguuugug

SEQ ID NO: 60 - RNA, 19 nt, Artificial
  RNA for silencing
cgguguuugugcagauccu

SEQ ID NO: 61 - RNA, 19 nt, Artificial
  RNA for silencing
gcccaugaaagacgguaca

SEQ ID NO: 62 - RNA, 19 nt, Artificial
  RNA for silencing
agacgguacaagauauacu

SEQ ID NO: 63 - RNA, 19 nt, Artificial
  RNA for silencing
gauauacuggaauucgauc

SEQ ID NO: 64 - RNA, 19 nt, Artificial
  RNA for silencing
uucgaucuuugaaacuuga

SEQ ID NO: 65 - RNA, 19 nt, Artificial
  RNA for silencing
acuugacaugaacccaggc

SEQ ID NO: 66 - RNA, 19 nt, Artificial
  RNA for silencing
cccaggcacugguaucugg

SEQ ID NO: 67 - RNA, 19 nt, Artificial
  RNA for silencing
gacagugcugcaaaauugg

SEQ ID NO: 68 - RNA, 19 nt, Artificial
  RNA for silencing
aauuggcucaaacagccug

SEQ ID NO: 69 - RNA, 19 nt, Artificial
  RNA for silencing
uuggcucaaacagccugaa

SEQ ID NO: 70 - RNA, 19 nt, Artificial
  RNA for silencing
acagccugaauccaauuua

SEQ ID NO: 71 - RNA, 19 nt, Artificial
  RNA for silencing
uccaauuuaggcaucgaaa

SEQ ID NO: 72 - RNA, 19 nt, Artificial
  RNA for silencing
uuuaggcaucgaaauaaaa

SEQ ID NO: 73 - RNA, 19 nt, Artificial
  RNA for silencing
auaaaagcuuuugaugaga

SEQ ID NO: 74 - RNA, 19 nt, Artificial
  RNA for silencing
aagcuuuugaugagacugg

SEQ ID NO: 75 - RNA, 19 nt, Artificial
  RNA for silencing
gcuuuugaugagacuggac

SEQ ID NO: 76 - RNA, 19 nt, Artificial
  RNA for silencing
gauggauugaacccauuuu

SEQ ID NO: 77 - RNA, 19 nt, Artificial
  RNA for silencing
cccauuuuuagaggucaga

SEQ ID NO: 78 - RNA, 19 nt, Artificial
  RNA for silencing
acggucccgcagagauuuu

SEQ ID NO: 79 - RNA, 19 nt, Artificial
  RNA for silencing
cggaaucccgauguugucg

SEQ ID NO: 80 - RNA, 19 nt, Artificial
  RNA for silencing
uccagucccauccaaaagc

SEQ ID NO: 81 - RNA, 19 nt, Artificial
  RNA for silencing
gcuuuuggaugggacugga

SEQ ID NO: 82 - RNA, 19 nt, Artificial
  RNA for silencing
aagauacaaagccaauuac

SEQ ID NO: 83 - RNA, 19 nt, Artificial
  RNA for silencing
gauacaaagccaauuacug

SEQ ID NO: 84 - RNA, 19 nt, Artificial
  RNA for silencing
agccaauuacugcuccgga

SEQ ID NO: 85 - RNA, 19 nt, Artificial
  RNA for silencing
uuacugcuccggagaaugc

SEQ ID NO: 86 - RNA, 19 nt, Artificial
  RNA for silencing
ugcgaauuuguguuucuac

SEQ ID NO: 87 - RNA, 19 nt, Artificial
  RNA for silencing
caggugagugugcggguau

SEQ ID NO: 88 - RNA, 19 nt, Artificial
  RNA for silencing
auacccgcacacucaccug

SEQ ID NO: 89 - RNA, 19 nt, Artificial
  RNA for silencing
gcaaaucccagagguccag

SEQ ID NO: 90 - RNA, 19 nt, Artificial
  RNA for silencing
aucccagagguccagcagg

SEQ ID NO: 91 - RNA, 19 nt, Artificial
  RNA for silencing
gauguccccuauaaacaug

SEQ ID NO: 92 - RNA, 19 nt, Artificial
  RNA for silencing
acaugcuguauuucaaugg

SEQ ID NO: 93 - RNA, 19 nt, Artificial
  RNA for silencing
uggaaaagaacaaauaaua

SEQ ID NO: 94 - RNA, 19 nt, Artificial
  RNA for silencing
aagaacaaauaauauaugg

SEQ ID NO: 95 - RNA, 19 nt, Artificial
  RNA for silencing
gaacaaauaauauauggaa

SEQ ID NO: 96 - RNA, 19 nt, Artificial
  RNA for silencing
caaauaauauauggaaaga

SEQ ID NO: 97 - RNA, 19 nt, Artificial
  RNA for silencing
auaauauauggaaagauac

SEQ ID NO: 98 - RNA, 19 nt, Artificial
  RNA for silencing
uauauggaaagauaccagc

SEQ ID NO: 99 - DNA, 18 bp, Artificial
  Oligonucleotide primer
cgaagaaccgagcgctgc

SEQ ID NO: 100 - DNA, 85 bp, Artificial
  Oligonucleotide primer
gggctcgagttccaaaaaagcgcagtgttactccacttctcttgaaagtggagtaacactgcgctgaataccgcttcctcctgag

SEQ ID NO: 101 - DNA, 20 bp, Artificial
  Oligonucleotide primer
gaattgtgggacggcggaag

SEQ ID NO: 102 - DNA, 83 bp, Artificial
  Oligonucleotide primer
ctcgagttccaaaaaagctgaccctgaagttcatctctcttgaagatgaacttcagggtcagcaaaccccagtgtctctcgga

SEQ ID NO: 103 - DNA, 83 bp, Artificial
  Oligonucleotide primer
ctcgagttccaaaaaaccagttgtcaagaagagcatctcttgaatgctcttcttgacaactggaaaccccagtgtctctcgga

SEQ ID NO: 104 - DNA, 83 bp, Artificial
  Oligonucleotide primer
ctcgagttccaaaaaaggatgctcattcaggacattctcttgaaatgtcctgaatgagcatccaaaccccagtgtctctcgga

SEQ ID NO: 105 - DNA, 83 bp, Artificial
  Oligonucleotide primer
ctcgagttccaaaaaaccctgtatccttactataatctcttgaattatagtaaggatacagggaaaccccagtgtctctcgga

SEQ ID NO: 106 - DNA, 83 bp, Artificial
  Oligonucleotide primer
ctcgagttccaaaaaagccactgagtcttcctcaatctcttgaattgaggaagactcagtggcaaaccccagtgtctctcgga

SEQ ID NO: 107 - DNA, 83 bp, Artificial
  Oligonucleotide primer
ctcgagttccaaaaaaccagcaacatacatgtcaatctcttgaattgacatgtatgttgctggaaaccccagtgtctctcgga

SEQ ID NO: 108 - DNA, 83 bp, Artificial
  Oligonucleotide primer
ctcgagttccaaaaaacctgcgtcacacagatacttctcttgaaagtatctgtgtgacgcaggaaaccccagtgtctctcgga

SEQ ID NO: 109 - DNA, 83 bp, Artificial
  Oligonucleotide primer
ctcgagttccaaaaaagctgaccctgaagttcatctctcttgaagatgaacttcagggtcagcgaatatctctacctcctagg

SEQ ID NO: 110 - DNA, 83 bp, Artificial
  Oligonucleotide primer
ctcgagttccaaaaaattcctgcagtggaggagcttctcttgaaagctcctccactgcaggaagaatatctctacctcctagg

SEQ ID NO: 111 - DNA, 83 bp, Artificial
  Oligonucleotide primer
ctcgagttccaaaaaatccagtcccatccaaaagctctcttgaagcttttggatgggactggagaatatctctacctcctagg

SEQ ID NO: 112 - DNA, 83 bp, Artificial
  Oligonucleotide primer
ctcgagttccaaaaaacaggtgagtgtgcgggtattctcttgaaatacccgcacactcacctggaatatctctacctcctagg

SEQ ID NO: 113 - RNA, 19 nt, Artificial Sequence
  RNA for silencing
ccagaauagaagccauaaa

SEQ ID NO: 114 - RNA, 19 nt, Artificial Sequence
  RNA for silencing
gcacaauuauggauauacu

SEQ ID NO: 115 - RNA, 19 nt, Artificial Sequence
  RNA for silencing
guacaagauauacuggaau

SEQ ID NO: 116 - RNA, 19 nt, Artificial Sequence
  RNA for silencing
ccuauaaacaugcuguauu

SEQ ID NO: 117 - RNA, 19 nt, Artificial Sequence
  RNA for silencing
gcgaauuuguguuucuaca

SEQ ID NO: 118 - RNA, 19 nt, Artificial Sequence
  RNA for silencing
gaguauugaugugaagaca

SEQ ID NO: 119 - RNA, 19 nt, Artificial Sequence
  RNA for silencing
ccuccagaauagaagccau

SEQ ID NO: 120 - RNA, 19 nt, Artificial Sequence
  RNA for silencing
ggucagaguuacagacaca

SEQ ID NO: 121 - RNA, 19 nt, Artificial Sequence
  RNA for silencing
caguggauuucgaagcuuu

SEQ ID NO: 122 - RNA, 19 nt, Artificial Sequence
  RNA for silencing
caacgguguuugugcagau

(19) United States
(12) Patent Application Publication
(10) Pub. No.: US 2013/0067606 A1
(43) Pub. Date: Mar. 14, 2013

(54) MODULATING PRODUCTION TRAITS IN AVIANS

(71) Applicants: Timothy James DORAN, Ocean Grove (AU); Robert John MOORE, Ascot Vale (AU); John William LOWENTHAL, Belmont (AU); Craig Allen SMITH, Prahran (AU); Andrew Henrik SINCLAIR, South Melbourne (AU)

(72) Inventors: Timothy James DORAN, Ocean Grove (AU); Robert John MOORE, Ascot Vale (AU); John William LOWENTHAL, Belmont (AU); Craig Allen SMITH, Prahran (AU); Andrew Henrik SINCLAIR, South Melbourne (AU)

(73) Assignees: Commonwealth Scientific and Industrial Research Organisation, Campbell (AU); AUSTRALIAN POULTRY CRC PTY LTD, Armidale (AU)

(21) Appl. No.: 13/602,927

(22) Filed: Sep. 4, 2012

Related U.S. Application Data
(63) Continuation of application No. 12/664,097, filed on Aug. 20, 2010, now abandoned.
(60) Provisional application No. 60/943,708, filed on Jun. 13, 2007.

(51) Int. Cl. A01K 67/027; C12N 15/113; C12N 5/10; C12N 1/21; C12N 1/15; A61K 31/713; C12N 15/63

(52) U.S. Cl. 800/8; 514/44 A; 536/24.5; 435/320.1; 435/252.3; 435/254.11; 435/419; 435/325

(57) ABSTRACT

The present invention relates to methods of modulating traits, particularly production traits, in avians such as chickens. In particular, the invention relates to the in ovo delivery of a dsRNA molecule, especially siRNAs, to modify production traits in commercially important birds.

### CROSS-REFERENCE TO RELATED APPLICATIONS

This application is a continuation of U.S. patent application Ser. No. 12/664,097, filed Aug. 20, 2010, which is the U.S. National Stage of International Application No. PCT/AU2008/000835, filed Jun. 12, 2008, which claims the benefit of U.S. Provisional Patent Application No. 60/943,708, filed Jun. 13, 2007; all of which are hereby incorporated by reference to the extent not inconsistent with the disclosure herewith.

### FIELD OF THE INVENTION

The present invention relates to methods of modulating traits, particularly production traits, in avians such as chickens. In particular, the invention relates to the in ovo delivery of a dsRNA molecule, especially siRNAs, to modify production traits in commercially important birds.

### BACKGROUND OF THE INVENTION

Man has modified the phenotypic characteristics of domestic animals through selection of seed stock over many generations ever since animals were domesticated. This has led to improvements in quantitative production parameters such as body size and muscle mass. More recent innovations of modifying production traits of poultry and/or improving resistance to pathogens has focussed on transgenic approaches, however, many consumers have concerns about genetically modified organisms.

Chicken producers have been searching for an efficient, economical method of determining the sex of day old chicks. Vent sexing and feather sexing have been used by the various producers, but these methods have been found to have substantial economic disadvantages because of the substantial time required and labour costs in separating the male from the female chicks. The use of probes (U.S. Pat. No. 5,508,165) is also an expensive procedure and not practical economically. Light sensing of anal areas of chicks (U.S. Pat. No. 4,417,663) is another way of determining sex of chicks, but it is also expensive and time consuming as each chick must be handled and manipulated. The use of experts who could feather sex the chicks has been used, but such experts are costly and feathering is time consuming.

There is a need for methods of modifying production traits in poultry that do not result in transformation of the bird's genome, but are amenable to high throughout processing.

### SUMMARY OF THE INVENTION

The present inventors have surprisingly found that administering a suitable nucleic acid molecule comprising a double-stranded region to an egg of an avian can modify the phenotype of the developing embryo.

Thus, in a first aspect the present invention provides a method of modifying a trait of an avian, the method comprising administering to an avian egg at least one nucleic acid molecule comprising a double-stranded region, wherein the nucleic acid molecule results in a reduction in the level of at least one RNA molecule and/or protein in the egg.

In another aspect, the present invention provides a method of modifying a trait of an avian, the method comprising administering to an avian egg at least one RNA molecule comprising a double-stranded region, wherein the RNA molecule results in a reduction in the level of at least one RNA molecule and/or protein in the egg, and wherein the method does not comprise electroporating the egg.

In a further aspect, the present invention provides a method of modifying a trait of an avian, the method comprising administering to an avian egg at least one RNA molecule comprising a double-stranded region (dsRNA), wherein the RNA molecule results in a reduction in the level of at least one RNA molecule and/or protein in the egg, and wherein the RNA molecule is administered to the air sac, yolk sac or chorion allantoic fluid.

In a preferred embodiment, the nucleic acid molecule is dsRNA. More preferably, the dsRNA is a siRNA or a shRNA.

In a further preferred embodiment, the trait is a production trait. Examples of production traits include, but are not limited to, muscle mass or sex.

In an embodiment, the production trait is sex and the nucleic acid molecule reduces the level of a protein encoded by a DMRT1 gene.

In an embodiment, the production trait is sex and the nucleic acid molecule reduces the level of a protein encoded by a WPKCI (ASW) gene.

In another embodiment, the production trait is muscle mass and the nucleic acid molecule reduces the level of a protein encoded by a myostatin gene.

Preferably, the nucleic acid molecule is administered by injection.

The avian can be any species of the Class Aves. Examples include, but are not limited to, chickens, ducks, turkeys, geese, bantams and quails. In a particularly preferred embodiment, the avian is a chicken.

In a further aspect, the present invention provides an avian produced using a method of the invention.

In another aspect, the present invention provides a chicken produced using a method of the invention.

In yet a further aspect, the present invention provides an isolated and/or exogenous nucleic acid molecule comprising a double-stranded region which reduces the level of at least one RNA molecule and/or protein when administered to an avian egg.

Preferably, the nucleic acid molecule is a dsRNA molecule. More preferably, the dsRNA is a siRNA or a shRNA.

In an embodiment, the nucleic acid molecule reduces the level of a protein encoded by a DMRT1 gene or a myostatin gene.

Also provided is a vector encoding a nucleic acid molecule, or a single strand thereof, according to the invention. Such vectors can be used in a host cell or cell-free expression system to produce nucleic acid molecules useful for the method of the invention.

In another aspect, the present invention provides a host cell comprising an exogenous nucleic acid molecule, or a single strand thereof, of the invention and/or a vector of the invention.

In another aspect, the present invention provides a composition comprising a nucleic acid molecule, or a single strand thereof, of the invention, a vector of the invention, and/or a host cell of the invention.

In a further aspect, the present invention provides an avian egg comprising a nucleic acid molecule, or a single strand thereof, of the invention, a vector of the invention, and/or a host cell of the invention.

In another aspect, the present invention provides a kit comprising a nucleic acid molecule, or a single strand thereof, of the invention, a vector of the invention, a host cell of the invention, and/or a composition of the invention.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1—PCR for shRNA expression cassettes. Schematic representation of the PCR strategy used to produce shRNA expression vectors. PCR used forward primers paired with reverse primers comprising all shRNA components. All final PCR products consisted of a chicken U6 promoter, shRNA sense, loop, shRNA antisense, termination sequence and XhoI site.

FIG. 2—Testing selected shRNAs for knockdown of EGFP-Dmrt1 gene fusion expression. Mean fluorescence intensity for each transfection condition expressed relative to pEGFP-Dmrt1. Error bars indicate standard error calculated on each individual experiment completed in triplicate.

FIG. 3—Testing selected shRNAs for knockdown of EGFP-Gdf8 gene fusion expression. DF1 cells were transfected with: Panel 1, pEGFP-C alone; Panel 2, pEGFP-Gdf8 transcriptional fusion alone; Panels 3-6 pEGFP-Gdf8 with either pshEGFP or the specific Gdf8 shRNA expression plasmids pshGdf8-258, pshGdf8-913 and pshGdf8-1002. Microscopy was performed using a Leica DM LB Fluorescence Microscope (Leica Microsystems, Germany) and images were captured at 50× magnification using a Leica DC300F colour digital camera (Leica Microsystems, Germany) and Photoshop 7.0 imaging software (Adobe®).

### KEY TO THE SEQUENCE LISTING

SEQ ID NO:1—Chicken myostatin (Genbank NM_{—}001001461).
SEQ ID NO:2—Nucleotide sequence encoding chicken myostatin (Genbank NM_{—}001001461).
SEQ ID NO:3—Partial chicken DMRT1 protein sequence (Genbank AF123456).
SEQ ID NO:4—Partial nucleotide sequence encoding chicken DMRT1 (Genbank AF123456).
SEQ ID NO:5—Chicken WPKCI (ASW) (Genbank AF148455).
SEQ ID NO:6—Nucleotide sequence encoding chicken WPKCI (ASW) (Genbank AF148455).
SEQ ID NO:7—Nucleotide sequence of chicken U6-1 promoter.
SEQ ID NO:8—Nucleotide sequence of chicken U6-3 promoter.
SEQ ID NO:9—Nucleotide sequence of chicken U6-4 promoter.
SEQ ID NO:10—Nucleotide sequence of chicken 7SK promoter.
SEQ ID NO's 11 to 98 and 113 to 122—RNA sequences useful for the invention.
SEQ ID NO's 99 to 112—Oligonucleotide primers.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, avian biology, RNA interference, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors).

The term “avian” as used herein refers to any species, subspecies or race of organism of the taxonomic Class Aves, such as, but not limited to, such organisms as chicken, turkey, duck, goose, quail, pheasants, parrots, finches, hawks, crows and ratites including ostrich, emu and cassowary. The term includes the various known strains of Gallus gallus (chickens), for example, White Leghorn, Brown Leghorn, Barred-Rock, Sussex, New Hampshire, Rhode Island, Australorp, Cornish, Minorca, Amrox, California Gray, Italian Partidge-coloured, as well as strains of turkeys, pheasants, quails, duck, ostriches and other poultry commonly bred in commercial quantities.

As used herein, the term “egg” refers to a fertilized ovum that has been laid by a bird. Typically, avian eggs consist of a hard, oval outer eggshell, the “egg white” or albumen, the egg yolk, and various thin membranes. Furthermore, “in ovo” refers to in an egg.

The terms “reduces”, “reduction” or variations thereof as used herein refers to a measurable decrease in the amount of a target RNA and/or target protein in the egg when compared to an egg from the same species of avian, more preferably strain or breed of avian, and even more preferably the same bird, that has not been administered with a nucleic acid as defined herein. The term also refers to a measurable reduction in the activity of a target protein. Preferably a reduction in the level of a target RNA and/or target protein is at least about 10%. More preferably the reduction is at least about 20%, 30%, 40%, 50%, 60%, 80%, 90% and even more preferably, about 100%.

As used herein, the phrase “the nucleic acid molecule results in a reduction” or variations thereof refers to the presence of the nucleic acid molecule in the egg inducing degradation of homologous RNAs in the egg by the process known in the art as “RNA interference” or “gene silencing”. Furthermore, the nucleic acid molecule directly results in the reduction, and is not transcribed in ovo produce the desired effect.

The “at least one RNA molecule” can be any type of RNA present in, and/or produced by, an avian egg. Examples include, but are not limited to, mRNA, snRNA, microRNA and tRNA.

As used herein, the term “production trait” refers to any phenotype of an avain that has commercial value such as muscle mass, sex and nutritional content.

As used herein, the term “muscle mass” refers to the weight of muscle tissue. An increase in muscle mass can be determined by weighing the total muscle tissue of a bird which hatches from an egg treated as described herein when compared to a bird from the same species of avian, more preferably strain or breed of avian, and even more preferably the same bird, that has not been administered with a nucleic acid as defined herein. Alternatively, specific muscles such as breast and/or leg muscles can be used to identify an increase in muscle mass. Preferably, the methods of the invention increase muscle mass by at about least 1%, 2.5%, 5%, 7.5%, and even more preferably, about 10%.

A “variant” of a nucleic acid molecule of the invention includes molecules of varying sizes of, and/or with one or more different nucleotides, but which are still capable of being used to silence the target gene. For example, variants may comprise additional nucleotides (such as 1, 2, 3, 4, or more), or less nucleotides. Furthermore, a few nucleotides may be substituted without influencing the ability of the nucleic acid to silence the target gene. In an embodiment, the variant includes additional 5′ and/or 3′ nucleotides which are homologous to the corresponding target RNA molecule and/or which enhance the stability of the nucleic acid molecule. In another embodiment, the nucleic acid molecules have no more than 4, more preferably no more than 3, more preferably no more than 2, and even more preferably no more than 1, nucleotide differences when compared to the sequences provided herein. In a further embodiment, the nucleic acid molecules have no more than 2, and more preferably no more than 1, internal additional and/or deletional nucleotides when compared to the sequences provided herein.

By an “isolated nucleic acid molecule”, we mean a nucleic acid molecule which is at least partially separated from the nucleic acid molecule with which it is associated or linked in its native state. Preferably, the isolated nucleic acid molecule is at least 60% free, preferably at least 75% free, and most preferably at least 90% free from other components with which they are naturally associated. Furthermore, the term “polynucleotide” is used interchangeably herein with the term “nucleic acid”.

The term “exogenous” in the context of a nucleic acid molecule refers to the nucleic acid molecule when present in a cell, or in a cell-free expression system, in an altered amount. Preferably, the cell is a cell that does not naturally comprise the nucleic acid molecule. However, the cell may be a cell which comprises an exogenous nucleic acid molecule resulting in an increased amount of the nucleic acid molecule. An exogenous nucleic acid molecule of the invention includes nucleic acid molecules which have not been separated from other components of the recombinant cell, or cell-free expression system, in which it is present, and nucleic acid molecules produced in such cells or cell-free systems which are subsequently purified away from at least some other components.

### Gene Silencing

The terms “RNA interference”, “RNAi” or “gene silencing” refers generally to a process in which a double-stranded RNA (dsRNA) molecule reduces the expression of a nucleic acid sequence with which the double-stranded RNA molecule shares substantial or total homology. However, it has more recently been shown that gene silencing can be achieved using non-RNA double stranded molecules (see, for example, U.S. 20070004667).

RNA interference (RNAi) is particularly useful for specifically inhibiting the production of a particular RNA and/or protein. Although not wishing to be limited by theory, Waterhouse et al. (1998) have provided a model for the mechanism by which dsRNA (duplex RNA) can be used to reduce protein production. This technology relies on the presence of dsRNA molecules that contain a sequence that is essentially identical to the mRNA of the gene of interest or part thereof, in this case an mRNA encoding a polypeptide according to the invention. Conveniently, the dsRNA can be produced from a single promoter in a recombinant vector or host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules for the present invention is well within the capacity of a person skilled in the art, particularly considering Waterhouse et al. (1998), Smith et al. (2000), WO 99/32619, WO 99/53050, WO 99/49029 and WO 01/34815.

The present invention includes nucleic acid molecules comprising and/or encoding double-stranded regions for gene silencing. The nucleic acid molecules are typically RNA but may comprise DNA, chemically-modified nucleotides and non-nucleotides.

The double-stranded regions should be at least 19 contiguous nucleotides, for example about 19 to 23 nucleotides, or may be longer, for example 30 or 50 nucleotides, or 100 nucleotides or more. The full-length sequence corresponding to the entire gene transcript may be used. Preferably, they are about 19 to about 23 nucleotides in length.

The degree of identity of a double-stranded region of a nucleic acid molecule to the targeted transcript should be at least 90% and more preferably 95-100%. The % identity of a nucleic acid molecule is determined by GAP (Needleman and Wunsch, 1970) analysis (GCG program) with a gap creation penalty=5, and a gap extension penalty=0.3. Preferably, the two sequences are aligned over their entire length.

The nucleic acid molecule may of course comprise unrelated sequences which may function to stabilize the molecule.

The term “short interfering RNA” or “siRNA” as used herein refers to a nucleic acid molecule which comprises ribonucleotides capable of inhibiting or down regulating gene expression, for example by mediating RNAi in a sequence-specific manner, wherein the double stranded portion is less than 50 nucleotides in length, preferably about 19 to about 23 nucleotides in length. For example the siRNA can be a nucleic acid molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siRNA can be assembled from two separate oligonucleotides, where one strand is the sense strand and the other is the antisense strand, wherein the antisense and sense strands are self-complementary.

As used herein, the term siRNA is meant to be equivalent to other terms used to describe nucleic acid molecules that are capable of mediating sequence specific RNAi, for example micro-RNA (miRNA), short hairpin RNA (shRNA), short interfering oligonucleotide, short interfering nucleic acid (siNA), short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siRNA molecules of the invention can be used to epigenetically silence genes at both the post-transcriptional level or the pre-transcriptional level. In a non-limiting example, epigenetic regulation of gene expression by siRNA molecules of the invention can result from siRNA mediated modification of chromatin structure to alter gene expression.

Preferred small interfering RNA (‘siRNA”) molecules comprise a nucleotide sequence that is identical to about 19 to 23 contiguous nucleotides of the target mRNA. In an embodiment, the target mRNA sequence commences with the dinucleotide AA, comprises a GC-content of about 30-70% (preferably, 30-60%, more preferably 40-60% and more preferably about 45%-55%), and does not have a high percentage identity to any nucleotide sequence other than the target in the genome of the avain (preferably chickens) in which it is to be introduced, e.g., as determined by standard BLAST search.

By “shRNA” or “short-hairpin RNA” is meant an siRNA molecule where less than about 50 nucleotides, preferably about 19 to about 23 nucleotides, is base paired with a complementary sequence located on the same RNA molecule, and where said sequence and complementary sequence are separated by an unpaired region of at least about 4 to 15 nucleotides which forms a single-stranded loop above the stem structure created by the two regions of base complementarity. Examples of sequences of a single-stranded loops are 5′ UUCAAGAGA 3′ and 5′ UUUGUGUAG 3′.

Included shRNAs are dual or bi-finger and multi-finger hairpin dsRNAs, in which the RNA molecule comprises two or more of such stem-loop structures separated by single-stranded spacer regions.

There are well-established criteria for designing siRNAs (see, for example, Elbashire et al., 2001; Amarzguioui et al., 2004; Reynolds et al., 2004). Details can be found in the websites of several commercial vendors such as Ambion, Dharmacon, GenScript, and OligoEngine. Typically, a number of siRNAs have to be generated and screened in order to compare their effectiveness.

Once designed, the dsRNAs for use in the method of the present invention can be generated by any method known in the art, for example, by in vitro transcription, recombinantly, or by synthetic means. siRNAs can be generated in vitro by using a recombinant enzyme, such as T7 RNA polymerase, and DNA oligonucleotide templates, or can be prepared in vivo, for example, in cultured cells. In a preferred embodiment, the nucleic acid molecule is produced synthetically.

In addition, strategies have been described for producing a hairpin siRNA from vectors containing, for example, a RNA polymerase III promoter. Various vectors have been constructed for generating hairpin siRNAs in host cells using either an H1-RNA or an snU6 RNA promoter (see SEQ ID NO's 7 to 9). A RNA molecule as described above (e.g., a first portion, a linking sequence, and a second portion) can be operably linked to such a promoter. When transcribed by RNA polymerase III, the first and second portions form a duplexed stem of a hairpin and the linking sequence forms a loop. The pSuper vector (OligoEngines Ltd., Seattle, Wash.) also can be used to generate siRNA.

Modifications or analogs of nucleotides can be introduced to improve the properties of the nucleic acid molecules of the invention. Improved properties include increased nuclease resistance and/or increased ability to permeate cell membranes. Accordingly, the terms “nucleic acid molecule” and “double-stranded RNA molecule” includes synthetically modified bases such as, but not limited to, inosine, xanthine, hypoxanthine, 2-aminoadenine, 6-methyl-, 2-propyl- and other alkyl- adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

### Traits, Particularly Production Traits, and Genes Responsible Therefor

The methods of the invention can be used to modify any trait of an avian species, particularly traits determined or influenced whilst the embryo is developing in the egg. Preferred traits which can be modified include sex and muscle mass.

In an embodiment, the production trait is sex and the nucleic acid molecule reduces the level of a protein encoded by a DMRT1 gene. DMRT1 was the first molecule implicated in sex determination that shows sequence conservation between phyla. The avian homologue of DMRT1 is found on the Z (sex) chromosome of chickens and is differentially expressed in the genital ridges of male and female chicken embryos (Raymond et al., 1999; Smith et al., 1999). DMRT1 is one of the few genes thus far implicated in mammalian sex determination that appears to have a strictly gonadal pattern of expression (Raymond et al., 1999).

Examples of nucleic acid molecules that can be used to reduce the level of chicken DMRT1 protein include, but are not limited to, those which comprise at least one of the following nucleotide sequences:

(SEQ ID NO: 11)
CCAGUUGUCAAGAAGAGCA
(SEQ ID NO: 12)
GGAUGCUCAUUCAGGACAU
(SEQ ID NO: 13)
CCCUGUAUCCUUACUAUAA
(SEQ ID NO: 14)
GCCACUGAGUCUUCCUCAA
(SEQ ID NO: 15)
CCAGCAACAUACAUGUCAA
(SEQ ID NO: 16)
CCUGCGUCACACAGAUACU
(SEQ ID NO: 17)
GGAGUAGUUGUACAGGUUG
(SEQ ID NO: 18)
GACUGGCUUGACAUGUAUG
(SEQ ID NO: 19)
AUGGCGGUUCUCCAUCCCU,

or a variant of any one thereof.

In a particularly preferred embodiment, the nucleic acid molecules that can be used to reduce the level of chicken DMRT1 protein comprises the sequence GCCACUGAGUCUUCCUCAA (SEQ ID NO:14), or a variant thereof.

A further example of a gene that can be targeted to modify sex as a production trait is the WPKCI gene. The avian gene WPKCI has been shown to be conserved widely on the avian W chromosome and expressed actively in the female chicken embryo before the onset of gonadal differentiation. It is suggested that WPKCI may play a role in the differentiation of the female gonad by interfering with the function of PKCI or by exhibiting its unique function in the nucleus (Hori et al., 2000). This gene has also been identified as ASW (avian sex-specific W-linked) (O'Neill et al., 2000).

In another embodiment, the production trait is muscle mass and the nucleic acid molecule reduces the level of a protein encoded by a myostatin gene. Myostatin, also termed “Growth and Differentiation Factor-8” (GDF8), is a recently discovered member of the TGFIβ super-family. Myostatin mRNA and protein have been shown to be expressed in skeletal muscle, heart and mammary gland. Targeted disruption of the myostatin gene in mice and a mutation in the third exon of myostatin gene in double-muscled Belgian Blue cattle, where a nonfunctional myostatin protein is expressed, leads to increased muscle mass. Hence, myostatin is a negative regulator of skeletal muscle growth.

Examples of nucleic acid molecules that can be used to reduce the level of chicken myostatin protein include, but are not limited to, those which comprise at least one of the following nucleotide sequences:

(SEQ ID NO: 20)
AAGCUAGCAGUCUAUGUUU
(SEQ ID NO: 21)
GCUAGCAGUCUAUGUUUAU
(SEQ ID NO: 22)
CGCUGAAAAAGACGGACUG
(SEQ ID NO: 23)
AAAGACGGACUGUGCAAUG
(SEQ ID NO: 24)
AGACGGACUGUGCAAUGCU
(SEQ ID NO: 25)
UGCUUGUACGUGGAGACAG
(SEQ ID NO: 26)
UACAAAAUCCUCCAGAAUA
(SEQ ID NO: 27)
AAUCCUCCAGAAUAGAAGC
(SEQ ID NO: 28)
UCCUCCAGAAUAGAAGCCA
(SEQ ID NO: 29)
UAGAAGCCAUAAAAAUUCA
(SEQ ID NO: 30)
GCCAUAAAAAUUCAAAUCC
(SEQ ID NO: 31)
AAAUUCAAAUCCUCAGCAA
(SEQ ID NO: 32)
AUUCAAAUCCUCAGCAAAC
(SEQ ID NO: 33)
AUCCUCAGCAAACUGCGCC
(SEQ ID NO: 34)
ACUGCGCCUGGAACAAGCA
(SEQ ID NO: 35)
CAAGCACCUAACAUUAGCA
(SEQ ID NO: 36)
GCACCUAACAUUAGCAGGG
(SEQ ID NO: 37)
CAUUAGCAGGGACGUUAUU
(SEQ ID NO: 38)
GCAGCUUUUACCCAAAGCU
(SEQ ID NO: 39)
UUCCUGCAGUGGAGGAGCU
(SEQ ID NO: 40)
CUGAUUGAUCAGUAUGAUG
(SEQ ID NO: 41)
GACGAUGACUAUCAUGCCA
(SEQ ID NO: 42)
CCGAGACGAUUAUCACAAU
(SEQ ID NO: 43)
UGCCUACGGAGUCUGAUUU
(SEQ ID NO: 44)
AUGGAGGGAAAACCAAAAU
(SEQ ID NO: 45)
AACCAAAAUGUUGCUUCUU
(SEQ ID NO: 46)
CCAAAAUGUUGCUUCUUUA
(SEQ ID NO: 47)
AAUGUUGCUUCUUUAAGUU
(SEQ ID NO: 48)
UGUUGCUUCUUUAAGUUUA
(SEQ ID NO: 49)
GUUUAGCUCUAAAAUACAA
(SEQ ID NO: 50)
AAUACAAUAUAACAAAGUA
(SEQ ID NO: 51)
UACAAUAUAACAAAGUAGU
(SEQ ID NO: 52)
UAUAACAAAGUAGUAAAGG
(SEQ ID NO: 53)
CAAAGUAGUAAAGGCACAA
(SEQ ID NO: 54)
AGUAGUAAAGGCACAAUUA
(SEQ ID NO: 55)
AGGCACAAUUAUGGAUAUA
(SEQ ID NO: 56)
UUAUGGAUAUACUUGAGGC
(SEQ ID NO: 57)
GUCCAAAAACCUACAACGG
(SEQ ID NO: 58)
AAACCUACAACGGUGUUUG
(SEQ ID NO: 59)
ACCUACAACGGUGUUUGUG
(SEQ ID NO: 60)
CGGUGUUUGUGCAGAUCCU
(SEQ ID NO: 61)
GCCCAUGAAAGACGGUACA
(SEQ ID NO: 62)
AGACGGUACAAGAUAUACU
(SEQ ID NO: 63)
GAUAUACUGGAAUUCGAUC
(SEQ ID NO: 64)
UUCGAUCUUUGAAACUUGA
(SEQ ID NO: 65)
ACUUGACAUGAACCCAGGC
(SEQ ID NO: 66)
CCCAGGCACUGGUAUCUGG
(SEQ ID NO: 67)
GACAGUGCUGCAAAAUUGG
(SEQ ID NO: 68)
AAUUGGCUCAAACAGCCUG
(SEQ ID NO: 69)
UUGGCUCAAACAGCCUGAA
(SEQ ID NO: 70)
ACAGCCUGAAUCCAAUUUA
(SEQ ID NO: 71)
UCCAAUUUAGGCAUCGAAA
(SEQ ID NO: 72)
UUUAGGCAUCGAAAUAAAA
(SEQ ID NO: 73)
AUAAAAGCUUUUGAUGAGA
(SEQ ID NO: 74)
AAGCUUUUGAUGAGACUGG
(SEQ ID NO: 75)
GCUUUUGAUGAGACUGGAC
(SEQ ID NO: 76)
GAUGGAUUGAACCCAUUUU
(SEQ ID NO: 77)
CCCAUUUUUAGAGGUCAGA
(SEQ ID NO: 78)
ACGGUCCCGCAGAGAUUUU
(SEQ ID NO: 79)
CGGAAUCCCGAUGUUGUCG
(SEQ ID NO: 80)
UCCAGUCCCAUCCAAAAGC
(SEQ ID NO: 81)
GCUUUUGGAUGGGACUGGA
(SEQ ID NO: 82)
AAGAUACAAAGCCAAUUAC
(SEQ ID NO: 83)
GAUACAAAGCCAAUUACUG
(SEQ ID NO: 84)
AGCCAAUUACUGCUCCGGA
(SEQ ID NO: 85)
UUACUGCUCCGGAGAAUGC
(SEQ ID NO: 86)
UGCGAAUUUGUGUUUCUAC
(SEQ ID NO: 87)
CAGGUGAGUGUGCGGGUAU
(SEQ ID NO: 88)
AUACCCGCACACUCACCUG
(SEQ ID NO: 89)
GCAAAUCCCAGAGGUCCAG
(SEQ ID NO: 90)
AUCCCAGAGGUCCAGCAGG
(SEQ ID NO: 91)
GAUGUCCCCUAUAAACAUG
(SEQ ID NO: 92)
ACAUGCUGUAUUUCAAUGG
(SEQ ID NO: 93)
UGGAAAAGAACAAAUAAUA
(SEQ ID NO: 94)
AAGAACAAAUAAUAUAUGG
(SEQ ID NO: 95)
GAACAAAUAAUAUAUGGAA
(SEQ ID NO: 96)
CAAAUAAUAUAUGGAAAGA
(SEQ ID NO: 97)
AUAAUAUAUGGAAAGAUAC
(SEQ ID NO: 98)
UAUAUGGAAAGAUACCAGC
(SEQ ID NO: 113)
CCAGAAUAGAAGCCAUAAA
(SEQ ID NO: 114)
GCACAAUUAUGGAUAUACU
(SEQ ID NO: 115)
GUACAAGAUAUACUGGAAU
(SEQ ID NO: 116)
CCUAUAAACAUGCUGUAUU
(SEQ ID NO: 117)
GCGAAUUUGUGUUUCUACA
(SEQ ID NO: 118)
GAGUAUUGAUGUGAAGACA
(SEQ ID NO: 119)
CCUCCAGAAUAGAAGCCAU
(SEQ ID NO: 120)
GGUCAGAGUUACAGACACA
(SEQ ID NO: 121)
CAGUGGAUUUCGAAGCUUU
(SEQ ID NO: 122)
CAACGGUGUUUGUGCAGAU,

or a variant of any one thereof.

In a particularly preferred embodiment, the nucleic acid molecules that can be used to reduce the level of chicken myostatin protein comprises the sequence CAGGUGAGUGUGCGGGUAU (SEQ ID NO:87), or a variant thereof.

### Vectors and Host Cells

The present invention also provides a vector encoding a nucleic acid molecule comprising a double-stranded region, or single strand thereof, of the present invention. Preferably, the vector is an expression vector capable of expressing the open reading frame(s) encoding a dsRNA in a host cell and/or cell free system. The host cell can be any cell type such as, not limited to, bacterial, fungal, plant or animal cells.

Typically, a vector of the invention will comprise a promoter operably linked to an open reading frame encoding a nucleic acid molecule of the invention, or a strand thereof.

As used herein, the term “promoter” refers to a nucleic acid sequence which is able to direct transcription of an operably linked nucleic acid molecule and includes, for example, RNA polymerase II and RNA polymerase III promoters. Also included in this definition are those transcriptional regulatory elements (e.g., enhancers) that are sufficient to render promoter-dependent gene expression controllable in a cell type-specific, tissue-specific, or temporal-specific manner, or that are inducible by external agents or signals.

“Operably linked” as used herein refers to a functional relationship between two or more nucleic acid (e.g., DNA) segments. Typically, it refers to the functional relationship of a transcriptional regulatory element to a transcribed sequence. For example, a promoter is operably linked to a coding sequence, such as an open reading frame encoding a double-stranded RNA molecule defined herein, if it stimulates or modulates the transcription of the coding sequence in an appropriate cell. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

By “RNA polymerase III promoter” or “RNA pol III promoter” or “polymerase III promoter” or “pol III promoter” is meant any invertebrate, vertebrate, or mammalian promoter, e.g., chicken, human, murine, porcine, bovine, primate, simian, etc. that, in its native context in a cell, associates or interacts with RNA polymerase III to transcribe its operably linked gene, or any variant thereof, natural or engineered, that will interact in a selected host cell with an RNA polymerase III to transcribe an operably linked nucleic acid sequence. By U6 promoter (e.g., chicken U6, human U6, murine U6), H1 promoter, or 7SK promoter is meant any invertebrate, vertebrate, or mammalian promoter or polymorphic variant or mutant found in nature to interact with RNA polymerase III to transcribe its cognate RNA product, i.e., U6 RNA, H1 RNA, or 7SK RNA, respectively. Examples of suitable promoters include cU6-1 (SEQ ID NO:7), cU6-3 (SEQ ID NO:8), cU6-4 (SEQ ID NO:9) and c7SK (SEQ ID NO:10).

When E. coli is used as a host cell, there is no limitation other than that the vector should have an “ori” to amplify and mass-produce the vector in E. coli (e.g., JM109. DH5α, HB101, or XL1B1ue), and a marker gene for selecting the transformed E. coli (e.g., a drug-resistance gene selected by a drug such as ampicillin, tetracycline, kanamycin, or chloramphenicol). For example, M13-series vectors, pUC-series vectors, pBR322, pBluescript, pCR-Script, and such can be used. pGEM-T, pDIRECT, pT7, and so on can also be used for subcloning and excision of the gene encoding the dsRNA as well as the vectors described above.

With regard to expression vectors for use in E. coli, such vectors include JM109, DH5α, HB101, or XL1 Blue, the vector should have a promoter such as lacZ promoter, araB promoter, or T7 promoter that can efficiently promote the expression of the desired gene in E. coli. Other examples of the vectors are “QIAexpress system” (Qiagen), pEGFP, and pET (for this vector, BL21, a strain expressing T7 RNA polymerase, is preferably used as the host).

In addition to the vectors for E. coli, for example, the vector may be a mammal-derived expression vector (e.g., pcDNA3 (Invitrogen), pEGF-BOS, pEF, and pCDM8), an insect cell-derived expression vector (e.g., “Bac-to-BAC baculovairus expression system” (GibcoBRL) and pBacPAK8), a plant-derived expression vector (e.g., pMH1 and pMH2), an animal virus-derived expression vector (e.g., pHSV, pMV, and pAdexLcw), a retrovirus-derived expression vector (e.g., pZIPneo), a yeast-derived expression vector (e.g., “Pichia Expression Kit” (Invitrogen), pNV11, and SP-Q01), a Bacillus subtilis-derived expression vector (e.g., pPL608 and pKTH50).

In order to express nucleic acid molecules in animal cells, such as CHO, COS, Vero and NIH3T3 cells, the vector should have a promoter necessary for expression in such cells, e.g., SV40 promoter, MMLV-LTR promoter, EF1α promoter, CMV promoter, etc., and more preferably it has a marker gene for selecting transformants (for example, a drug resistance gene selected by a drug (e.g., neomycin. G418, etc.)). Examples of vectors with these characteristics include pMAM, pDR2, pBK-RSV, pBK-CMV, pOPRSV and pOP13.

Nucleic acid molecules comprising a double-stranded region of the present invention can be expressed in animals by, for example, inserting an open reading frame(s) encoding the nucleic acid into an appropriate vector and introducing the vector by the retrovirus method, liposome method, cationic liposome method, adenovirus method, and so on. The vectors used include, but are not limited to, adenoviral vectors (e.g., pAdexlcw) and retroviral vectors (e.g., pZIPneo). General techniques for gene manipulation, such as insertion of nucleic acids of the invention into a vector, can be performed according to conventional methods.

The present invention also provides a host cell into which an exogenous nucleic acid molecule, typically in a vector of the present invention, has been introduced. The host cell of this invention can be used as, for example, a production system for producing or expressing the nucleic acid molecule. For in vitro production, eukaryotic cells or prokaryotic cells can be used. Useful eukaryotic host cells may be animal, plant, or fungi cells. As animal cells, mammalian cells such as CHO, COS, 3T3, myeloma, baby hamster kidney (BHK), HeLa, or Vero cells MDCK cells, DF1 cells, amphibian cells such as Xenopus oocytes, or insect cells such as Sf9, Sf21, or Tn5 cells can be used. CHO cells lacking DHFR gene (dhfr-CHO) or CHO K-1 may also be used. The vector can be introduced into the host cell by, for example, the calcium phosphate method, the DEAE-dextran method, cationic liposome DOTAP (Boehringer Mannheim) method, electroporation, lipofection, etc.

Useful prokaryotic cells include bacterial cells, such as E. coli, for example, JM109, DH5a, and HB101, or Bacillus subtilis.

Culture medium such as DMEM, MEM, RPMI-1640, or IMDM may be used for animal cells. The culture medium can be used with or without serum supplement such as fetal calf serum (FCS). The pH of the culture medium is preferably between about 6 and 8. Cells are typically cultured at about 30 to 40° C. for about 15 to 200 hr, and the culture medium may be replaced, aerated, or stirred if necessary.

### Compositions

The present invention also provides compositions comprising a nucleic acid molecule comprising a double-stranded region that can be administered to an avian egg. A composition comprising a nucleic acid molecule comprising a double-stranded region may contain a pharmaceutically acceptable carrier to render the composition suitable for administration.

Suitable pharmaceutical carriers, excipients and/or diluents include, but are not limited to, lactose, sucrose, starch powder, talc powder, cellulose esters of alkonoic acids, magnesium stearate, magnesium oxide, crystalline cellulose, methyl cellulose, carboxymethyl cellulose, gelatin, glycerin, sodium alginate, antibacterial agents, antifungal agents, gum arabic, acacia gum, sodium and calcium salts of phosphoric and sulfuric acids, polyvinylpyrrolidone and/or polyvinyl alcohol, saline, and water. In an embodiment, the carrier, excipient and/or diluent is phosphate buffered saline or water.

The composition may also comprise a transfection promoting agent. Transfection promoting agents used to facilitate the uptake of nucleic acids into a living cell are well known within the art. Reagents enhancing transfection include chemical families of the types; polycations, dendrimers, DEAE Dextran, block copolymers and cationic lipids. Preferably, the transfection-promoting agent is a lipid-containing compound (or formulation), providing a positively charged hydrophilic region and a fatty acyl hydrophobic region enabling self-assembly in aqueous solution into vesicles generally known as micelles or liposomes, as well as lipopolyamines.

It is understood that any conventional media or agent may be used so long as it is not incompatible with the compositions or methods of the invention.

### Administration

Administration of a nucleic acid molecule comprising a double-stranded region (including a composition comprising a nucleic acid molecule comprising a double-stranded region) is conveniently achieved by injection into the egg, and generally injection into the air sac. Notwithstanding that the air sac is the preferred route of in ovo administration, other regions such as the yolk sac or chorion allantoic fluid may also be inoculated by injection. The hatchability rate might decrease slightly when the air sac is not the target for the administration although not necessarily at commercially unacceptable levels. The mechanism of injection is not critical to the practice of the present invention, although it is preferred that the needle does not cause undue damage to the egg or to the tissues and organs of the developing embryo or the extra-embryonic membranes surrounding the embryo.

When the production trait is sex, it is preferred that the nucleic acid molecule is administered within four days of the egg having been laid.

Generally, a hypodermic syringe fitted with an approximately 22 gauge needle is suitable. The method of the present invention is particularly well adapted for use with an automated injection system, such as those described in U.S. Pat. No. 4,903,635, U.S. Pat. No. 5,056,464, U.S. Pat. No. 5,136,979 and U.S. 20060075973.

The nucleic acid molecule is administered in an effective amount sufficient to at least some degree modify the target trait. With regard to sex, the modification can be detected comparing a suitable number of samples subjected to the method of the invention to a similar number that have not. Statistically significant variation in the sex of the birds between to the two groups will be indicative that an effective amount has been administered. Other means of determining an effective amount for sex or other traits is well within the capacity of those skilled in the art.

Preferably, about 1 ng to 100 μg, more preferably about 100 ng to 1 μg, of nucleic acid is administered to the egg. Furthermore, it is preferred that the nucleic acid to be administered is in a volume of about 1 μl to 1 ml, more preferably about 10 μl to 500 μl.

### EXAMPLES

### Example 1

### Identification of shRNA Molecules for Down-Regulating DMRT1 Protein Production in Chickens

### Selection of shRNA Sequences Targeting DMRT1

The present inventors selected 30 predicted siRNA sequences for Dmrt1 using the Ambion designed siRNA target finder (www.ambion.com/techlib/misc/siRNA_finder.html). The 30 siRNA sequences were then screened for selection of shRNAs (Table 1). There are several algorithms available to select potential siRNA sequences for specific target genes. It has been shown, however that many of these predicted siRNAs do not function effectively when processed from expressed shRNAs. Taxman et al. (2006) have specifically designed an algorithm to predict effective shRNA molecules and the inventors made their own modification to the algorithm to improve shRNA prediction. The inventors applied the modified Taxman algorithm to the 30 selected siRNAs so as to choose sequences for testing as shRNAs for the specific knockdown of Dmrt1 gene expression.

There are four criteria for shRNA selection using the Taxman algorithm. Three of the criteria are scored for out of a maximum number of 4 points. These criteria are: 1) C or G on the 5′ end of the sequence=1 point, A or T on 5′ end=−1 point; 2) A or T on the 3′ end=1 point, C or G on the 3′ end=−1 point; 3) 5 or more A or T in the seven 3′ bases=2 points, 4 A or T in the seven 3′ bases=1 point. shRNA sequences with the highest scores are preferred. The fourth criteria is based on a calculation for the free-energy of the 6 central bases of the shRNA sequence (bases 6-11 of the sense strand hybridised to bases 9-14 of the antisense strand). shRNAs with a central duplex ΔG>−12.9 kcal/mol are preferred. The modification to the Taxman algorithm the use different free-energy parameters for predictions of RNA duplex stability as published by Freier et al. (1986). Based on the algorithm, the inventors chose 6 of the siRNA target finder siRNA sequences as potentially effective shRNAs to test for their ability to knockdown Dmrt1 gene expression. The selected sequences are highlighted in bold in Table 1 and their 5′-3′ sequence is shown in Table 2. These 6 sequences were used to construct ddRNAi plasmids for the expression of the 6 shRNAs.

### Construction of ddRNAi Plasmids for Expression of Selected shRNAs

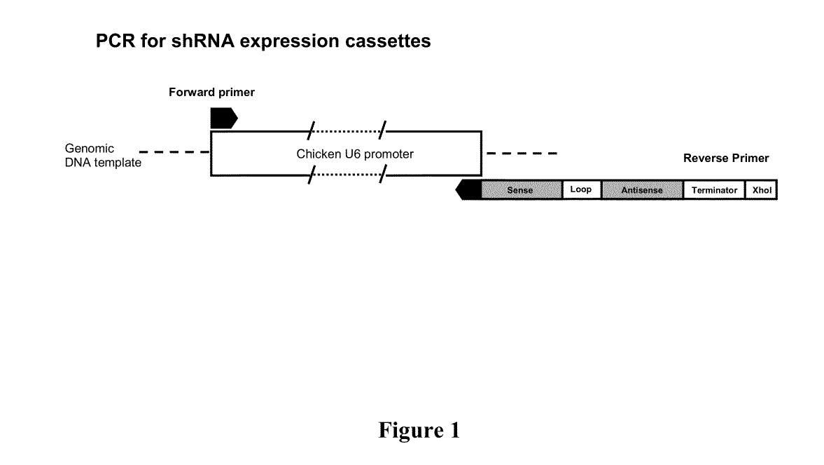

Chicken polymerase III promoters cU6-1 (GenBank accession number DQ531567) and cU6-4 (DQ531570) were used as templates to construct ddRNAi expression plasmids for the selected dmrt1 and control (EGFP and irrelevant) shRNAs, via a one-step PCR (FIG. 1). PCR for the construction of the shRNA plasmids used primer TD175 paired with TH346 (for shDmrt1-346), TH461 (shDmrt1-461), TH566 (shDmrt1-566), TH622 (shDmrt1-622), TH697 (shDmrt1-697), TH839 (shDmrt1-839) or TD195 (shEGFP) (see Table 3 for primer sequence and details of the specific shRNA amplified). The reverse primers in each PCR were designed to comprise the last 20 nt of each promoter sequence, shRNA sense, loop, and shRNA antisense sequence and were HPLC purified. Full-length amplified expression cassette products were ligated into pGEM-T Easy and then sequenced. The final shRNA expression plasmids used in gene knockdown assays were named pshDmrt1-346, pshDmrt1-461, pshDmrt1-566, pshDmrt1-622, pshDmrt1-697, pshDmrt1-839, and pshEGFP. A cU6-1 irrelevant control plasmid was also constructed and used for mock comparison in the gene expression assays (see below). For this mock plasmid, forward primer TD135 was paired with reverse primer TD149 comprising the last 20 nt of the chU6-1 promoter and all other irrelevant shRNA components. The PCR product was ligated into pGEM-T Easy and sequenced.

TABLE 1
Algorithm selection of shRNA sequences targeting Dmrt1.

| | 5’ end | | 3’ end | | |
|---|---|---|---|---|---|
| shRNA | score | ΔG central | score | A + T in 3’ | Score |
| Dmrt1-346 | 1 | −11.2 | 1 | 1 | 3 |
| Dmrt1-461 | 1 | −13.3 | 1 | 1 | 3 |
| Dmrt1-566 | 1 | −11.6 | 1 | 2 | 4 |
| Dmrt1-622 | 1 | −13.6 | 1 | 1 | 3 |
| Dmrt1-697 | 1 | −10.7 | 1 | 2 | 4 |
| Dmrt1-839 | 1 | −14.2 | 1 | 2 | 4 |
| Dmrt1-581 | 1 | −13.2 | −1 | 2 | 2 |
| Dmrt1-341 | 1 | −15.8 | 1 | 2 | 4 |
| Dmrt1-578 | −1 | −10.9 | 1 | 2 | 2 |
| Dmrt1-563 | 1 | −12.8 | 1 | 2 | 4 |
| Dmrt1-779 | −1 | −14 | 1 | 1 | 1 |
| Dmrt1-837 | 1 | −15.5 | 1 | 2 | 4 |
| Dmrt1-593 | 1 | −14.7 | −1 | 1 | 1 |
| Dmrt1-778 | 1 | −15.2 | −1 | 1 | 1 |
| Dmrt1-577 | −1 | −9.8 | 1 | 1 | 1 |
| Dmrt1-583 | 1 | −13.8 | 1 | 0 | 2 |
| Dmrt1-839 | 1 | −14.2 | 1 | 2 | 4 |
| Dmrt1-691 | 1 | −16.8 | −1 | 2 | 2 |
| Dmrt1-455 | 1 | −15.4 | −1 | 1 | 1 |
| Dmrt1-705 | −1 | −11.5 | −1 | 2 | 0 |
| Dmrt1-532 | 1 | −14.6 | 1 | 1 | 3 |
| Dmrt1-184 | 1 | −15.3 | 1 | 1 | 3 |
| Dmrt1-761 | −1 | −13.6 | 1 | 0 | 0 |
| Dmrt1-505 | −1 | −15 | 1 | 2 | 2 |
| Dmrt1-208 | 1 | −17.1 | 1 | 2 | 4 |
| Dmrt1-219 | 1 | −13.4 | −1 | 0 | 0 |
| Dmrt1-458 | 1 | −14.2 | 1 | 1 | 3 |
| Dmrt1-837 | 1 | −15.2 | 1 | 2 | 4 |
| Dmrt1-701 | 1 | −10.7 | 1 | 0 | 2 |
| Dmrt1-628 | 1 | −13.6 | 1 | 1 | 3 |

TABLE 2
Sequence of Dmrt1 shRNAs.

| shRNA | 5′ - 3′ Sequence |
|---|---|
| Dmrt1-346 | CCAGUUGUCAAGAAGAGCA (SEQ ID NO: 11) |
| Dmrt1-461 | GGAUGCUCAUUCAGGACAU (SEQ ID NO: 12) |
| Dmrt1-566 | CCCUGUAUCCUUACUAUAA (SEQ ID NO: 13) |
| Dmrt1-622 | GCCACUGAGUCUUCCUCAA (SEQ ID NO: 14) |
| Dmrt1-697 | CCAGCAACAUACAUGUCAA (SEQ ID NO: 15) |
| Dmrt1-839 | CCUGCGUCACACAGAUACU (SEQ ID NO: 16) |

TABLE 3
Sequence and details of primers used.

| Name | Sequence 5′-3′ | Location/Feature |
|---|---|---|
| TD135 | CGAAGAACCGAGCGCTGC (SEQ ID NO: 99) | cU6-1 |
| TD149 | GGGCTCGAGTTCCAAAAAAGCGCAGTGTTACTCCA | cU6-1 shIrr |
| | CTTCTCTTGAAAGTGGAGTAACACTGCGCTGAATA | |
| | CCGCTTCCTCCTGAG (SEQ ID NO: 100) | |
| TD175 | GAATTGTGGGACGGCGGAAG (SEQ ID NO: 101) | cU6-4 |
| TD195 | CTCGAGTTCCAAAAAAGCTGACCCTGAAGTTCATC | cU6-4 shEGFP |
| | TCTCTTGAAGATGAACTTCAGGGTCAGCAAACCCC | |
| | AGTGTCTCTCGGA (SEQ ID NO: 102) | |
| TH346 | CTCGAGTTCCAAAAAACCAGTTGTCAAGAAGAGCA | cU6-4 shDmrt1-346 |
| | TCTCTTGAATGCTCTTCTTGACAACTGGAAACCCCA | |
| | GTGTCTCTCGGA (SEQ ID NO: 103) | |
| TH461 | CTCGAGTTCCAAAAAAGGATGCTCATTCAGGACAT | cU6-4 shDmrt1-461 |
| | TCTCTTGAAATGTCCTGAATGAGCATCCAAACCCC | |
| | AGTGTCTCTCGGA (SEQ ID NO: 104) | |
| TH566 | CTCGAGTTCCAAAAAACCCTGTATCCTTACTATAAT | cU6-4 shDmrt1-566 |
| | CTCTTGAATTATAGTAAGGATACAGGGAAACCCCA | |
| | GTGTCTCTCGGA (SEQ ID NO: 105) | |
| TH622 | CTCGAGTTCCAAAAAAGCCACTGAGTCTTCCTCAA | cU6-4 shDmrt1-622 |
| | TCTCTTGAATTGAGGAAGACTCAGTGGCAAACCCC | |
| | AGTGTCTCTCGGA (SEQ ID NO: 106) | |
| TH697 | CTCGAGTTCCAAAAAACCAGCAACATACATGTCAA | cU6-4 shDmrt1-697 |
| | TCTCTTGAATTGACATGTATGTTGCTGGAAACCCCA | |
| | GTGTCTCTCGGA (SEQ ID NO: 107) | |
| TH839 | CTCGAGTTCCAAAAAACCTGCGTCACACAGATACT | cU6-4 shDmrt1-839 |
| | TCTCTTGAAAGTATCTGTGTGACGCAGGAAACCCC | |
| | AGTGTCTCTCGGA (SEQ ID NO: 108) | |

Each ddRNAi plasmid was constructed so that the start of each shRNA sequence was at the +1 position of the native U6 snRNA transcripts. A XhoI restriction enzyme site was engineered downstream of the termination signal to allow screening for full-length shRNA products inserted into pGEM-T Easy. All final shRNA expression vectors consisted of either one of the full length chicken U6 promoters, a shRNA sense sequence, a loop sequence, a shRNA antisense sequence, a termination sequence and a XhoI site. The loop sequence used in all shRNAs was 5′ UUCAAGAGA 3′.

Testing selected shRNAs for knockdown of Dmrt1 gene expression A reporter gene expression assay was used to test shRNAs for silencing of Dmrt1. The reporter gene was a transcriptional gene fusion of Dmrt1 inserted downstream of the 3′ end of the Enhanced Green Fluorescent Protein (EGFP) gene in pEGFP-C (Clontech). The reporter plasmid was constructed as follows: cDNA of Dmrt1 was reverse transcribed from total RNA isolated from 4 day old embryo's and cloned into the multiple cloning site of pCMV-Script (Stratagene). The Dmrt1 insert was removed from the cloning vector as a NotI-EcoRI fragment and cloned downstream of the EGFP gene in pEGFP-C (Clontech). The resulting plasmid was named pEGFP-Dmrt1. This plasmid was transfected into chicken DF-1 cells and expression of the transcriptional gene fusion was confirmed by measuring EGFP fluorescence using flow cytometry as described below. DF-1 cells are a continuous line of chicken embryo fibroblasts, derived from an EV-0 embryo (ATCC, CRL-12203), and hence are a model system for studying the in ovo effects of the RNAi molecules.

Dmrt1 gene silencing assays were conducted by co-transfecting DF-1 cells with the pEGFP-Dmrt1 reporter plasmid and each of the ddRNAi plasmids expressing the Dmrt1 specific and control shRNAs. The co-transfection experiments were performed as follows: DF-1 cells (ATCC CRL-12203, chicken fibroblast) were maintained in a humidified atmosphere containing 5% CO₂ at 37° C. in Dulbecco's Modified Eagle's Medium (DMEM) containing 4.5 g/l glucose, 1.5g/l sodium bicarbonate, 10% foetal calf serum (FCS), 2 mM L-glutamine supplemented with penicillin (100 U/ml) and streptomycin (100 μg/l). DF1 cells were passaged as required using 0.25% (w/v) trypsin-ethylenediaminetetraacetic acid (EDTA).

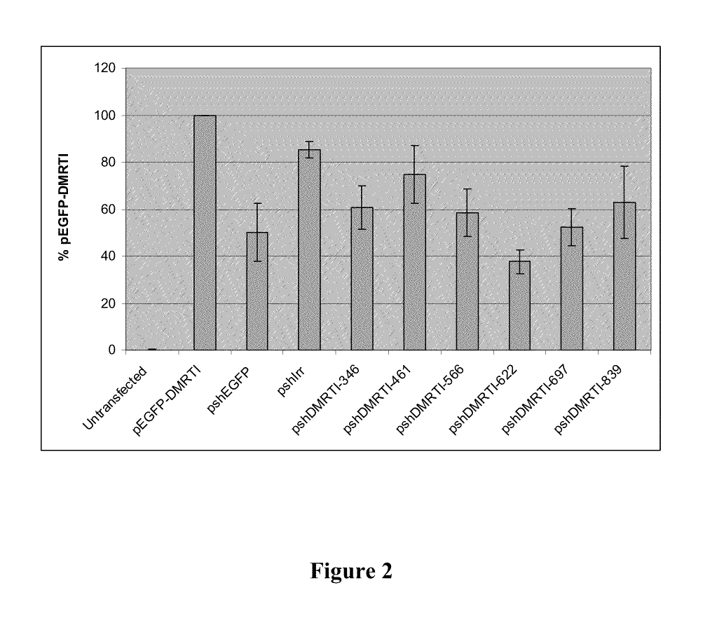

Co-transfection of pEGFP-Dmrt1 and ddRNAi plasmids for EGFP-Dmrt1 fusion silencing assays was conducted in DF-1 cells grown to 80-90% confluence, in 24 well culture plates (Nunc) for flow cytometry analysis. Cells were transfected with a total of 1 μg of plasmid DNA, per well, using Lipofectamine™2000 transfection reagent (Invitrogen). EGFP expression was analysed in transfected DF-1 cells at 60 hours post-transfection using flow cytometry analysis of transfections performed in triplicate. Cells were trypsinised using 100 μl of 0.25% trypsin-EDTA, pelleted at 2000 rpm for 5 minutes, washed once in 1 ml of cold phosphate buffered saline-A (PBSA) (Oxoid), twice in 1 ml of FACS-wash solution (PBSA+1% FCS) and resuspended in 250 μl of FACS-wash solution. Flow cytometry sampling was performed using a FACScalibur (Becton Dickinson) fluorescence activated cell sorter. Data acquisition and calculation of mean fluorescence intensity (MFI) values for triplicate co-transfection samples, was performed using CELLQuest software (Becton Dickinson). The results of the gene silencing assay are shown in FIG. 2. pshEGFP was used as a positive control. The shRNA expressed from this plasmid is known to effectively target the EGFP region of the fusion transcript and was shown to reduce reporter fluorescence by approximately 50%. Compared to the negative control irrelevant shRNA expressed from pshIrr, the Dmrt1 specific shRNAs were observed to knockdown expression of the reporter gene to varying levels. shDmrt1-622 induced the greatest level of gene silencing of approximately 60%.

### Example 2

### Identification of shRNA Molecules for Down-Regulating Myostatin Protein Production in Chickens

### Selection of shRNA Sequences Targeting Myostatin (Gdf8)

79 predicted siRNA sequences for Gdf8 were identified using the Ambion designed siRNA target finder (www.ambion.com/techlib/misc/siRNA_finder.html) (Table 4). Additional siRNA sequences optimized using the Taxman algorithm are provided in Table 5. The inventors selected 3 of these sequences (Gdf8-258, Gdf8-913 and Gdf8-1002) for the construction of ddRNAi plasmids for expression of shRNAs (shown in bold in Table 4).

### Construction of ddRNAi Plasmids for Expression of Selected shRNAs

The chicken polymerase III promoter cU6-1 (GenBank accession number DQ531567) was used as template to construct ddRNAi expression plasmids for the selected Gdf8 and cEGFP shRNAs, via a one-step PCR (FIG. 1). PCR for the construction of the shRNA plasmids used primer TD135 paired with DS304 (for shGdf8-253), DS305 (shGdf8-913), DS306 (shGdf8-1002) or TD148 (shEGFP) (see Table 6 for primer sequence and details of the specific shRNA amplified). The reverse primers in each PCR were designed to comprise the last 20 nt of each promoter sequence, shRNA sense, loop, and shRNA antisense sequence and were HPLC purified. Full-length amplified expression cassette products were ligated into pGEM-T Easy and then sequenced. The final shRNA expression plasmids used in gene knockdown assays were named pshGdf8-253, pshGdf8-913, pshGdf8-1002 and pshEGFP.

TABLE 4
Sequence of Gdf8 shRNAs.

| shRNA | 5′-3′ Sequence | shRNA | 5′-3′ Sequence |
|---|---|---|---|
| Gdf8-5 | AAGCUAGCAGUCUAUGUUU | Gdf8-555 | CGGUGUUUGUGCAGAUCCU |
| | (SEQ ID NO: 20) | | (SEQ ID NO: 60) |
| Gdf8-7 | GCUAGCAGUCUAUGUUUAU | Gdf8-567 | GCCCAUGAAAGACGGUACA |
| | (SEQ ID NO: 21) | | (SEQ ID NO: 61) |
| Gdf8-96 | CGCUGAAAAAGACGGACUG | Gdf8-578 | AGACGGUACAAGAUAUACU |
| | (SEQ ID NO: 22) | | (SEQ ID NO: 62) |
| Gdf8-103 | AAAGACGGACUGUGCAAUG | Gdf8-590 | GAUAUACUGGAAUUCGAUC |
| | (SEQ ID NO: 23) | | (SEQ ID NO: 63) |
| Gdf8-105 | AGACGGACUGUGCAAUGCU | Gdf8-603 | UUCGAUCUUUGAAACUUGA |
| | (SEQ ID NO: 24) | | (SEQ ID NO: 64) |
| Gdf8-120 | UGCUUGUACGUGGAGACAG | Gdf8-615 | ACUUGACAUGAACCCAGGC |
| | (SEQ ID NO: 25) | | (SEQ ID NO: 65) |
| Gdf8-144 | UACAAAAUCCUCCAGAAUA | Gdf8-654 | CCCAGGCACUGGUAUCUGG |
| | (SEQ ID NO: 26) | | (SEQ ID NO: 66) |
| Gdf8-149 | AAUCCUCCAGAAUAGAAGC | Gdf8-667 | GACAGUGCUGCAAAAUUGG |
| | (SEQ ID NO: 27) | | (SEQ ID NO: 67) |
| Gdf8-151 | UCCUCCAGAAUAGAAGCCA | Gdf8-669 | AAUUGGCUCAAACAGCCUG |
| | (SEQ ID NO: 28) | | (SEQ ID NO: 68) |
| Gdf8-161 | UAGAAGCCAUAAAAAUUCA | Gdf8-678 | UUGGCUCAAACAGCCUGAA |
| | (SEQ ID NO: 29) | | (SEQ ID NO: 69) |
| Gdf8-166 | GCCAUAAAAAUUCAAAUCC | Gdf8-688 | ACAGCCUGAAUCCAAUUUA |
| | (SEQ ID NO: 30) | | (SEQ ID NO: 70) |
| Gdf8-173 | AAAUUCAAAUCCUCAGCAA | Gdf8-696 | UCCAAUUUAGGCAUCGAAA |
| | (SEQ ID NO: 31) | | (SEQ ID NO: 71) |
| Gdf8-175 | AUUCAAAUCCUCAGCAAAC | Gdf8-709 | UUUAGGCAUCGAAAUAAAA |
| | (SEQ ID NO: 32) | | (SEQ ID NO: 72) |
| Gdf8-181 | AUCCUCAGCAAACUGCGCC | Gdf8-713 | AUAAAAGCUUUUGAUGAGA |
| | (SEQ ID NO: 33) | | (SEQ ID NO: 73) |
| Gdf8-195 | ACUGCGCCUGGAACAAGCA | Gdf8-715 | AAGCUUUUGAUGAGACUGG |
| | (SEQ ID NO: 34) | | (SEQ ID NO: 74) |
| Gdf8-208 | CAAGCACCUAACAUUAGCA | Gdf8-772 | GCUUUUGAUGAGACUGGAC |
| | (SEQ ID NO: 35) | | (SEQ ID NO: 75) |
| Gdf8-211 | GCACCUAACAUUAGCAGGG | Gdf8-783 | GAUGGAUUGAACCCAUUUU |
| | (SEQ ID NO: 36) | | (SEQ ID NO: 76) |
| Gdf8-219 | CAUUAGCAGGGACGUUAUU | Gdf8-822 | CCCAUUUUUAGAGGUCAGA |
| | (SEQ ID NO: 37) | | (SEQ ID NO: 77) |
| Gdf8-240 | GCAGCUUUUACCCAAAGCU | Gdf8-866 | ACGGUCCCGCAGAGAUUUU |
| | (SEQ ID NO: 38) | | (SEQ ID NO: 78) |
| Gdf8-258 | UUCCUGCAGUGGAGGAGC | Gdf8-871 | CGGAAUCCCGAUGUUGUCG |
| | U (SEQ ID NO: 39) | | (SEQ ID NO: 79) |
| Gdf8-277 | CUGAUUGAUCAGUAUGAU | Gdf8-913 | UCCAGUCCCAUCCAAAAGC |
| | G (SEQ ID NO: 40) | | (SEQ ID NO: 80) |
| Gdf8-334 | GACGAUGACUAUCAUGCCA | Gdf8-948 | GCUUUUGGAUGGGACUGGA |
| | (SEQ ID NO: 41) | | (SEQ ID NO: 81) |
| Gdf8-356 | CCGAGACGAUUAUCACAAU | Gdf8-950 | AAGAUACAAAGCCAAUUAC |
| | (SEQ ID NO: 42) | | (SEQ ID NO: 82) |
| Gdf8-406 | UGCCUACGGAGUCUGAUUU | Gdf8-957 | GAUACAAAGCCAAUUACUG |
| | (SEQ ID NO: 43) | | (SEQ ID NO: 83) |
| Gdf8-416 | AUGGAGGGAAAACCAAAA | Gdf8-963 | AGCCAAUUACUGCUCCGGA |
| | U (SEQ ID NO: 44) | | (SEQ ID NO: 84) |
| Gdf8-418 | AACCAAAAUGUUGCUUCUU | Gdf8-979 | UUACUGCUCCGGAGAAUGC |
| | (SEQ ID NO: 45) | | (SEQ ID NO: 85) |
| Gdf8-422 | CCAAAAUGUUGCUUCUUUA | Gdf8-985 | UGCGAAUUUGUGUUUCUAC |
| | (SEQ ID NO: 46) | | (SEQ ID NO: 86) |
| Gdf8-424 | AAUGUUGCUUCUUUAAGU | Gdf8-1002 | CAGGUGAGUGUGCGGGUA |
| | U (SEQ ID NO: 47) | | U (SEQ ID NO: 87) |
| Gdf8-441 | UGUUGCUUCUUUAAGUUU | Gdf8-1033 | AUACCCGCACACUCACCUG |
| | A (SEQ ID NO: 48) | | (SEQ ID NO: 88) |
| Gdf8-453 | GUUUAGCUCUAAAAUACAA | Gdf8-1037 | GCAAAUCCCAGAGGUCCAG |
| | (SEQ ID NO: 49) | | (SEQ ID NO: 89) |
| Gdf8-455 | AAUACAAUAUAACAAAGU | Gdf8-1081 | AUCCCAGAGGUCCAGCAGG |
| | A (SEQ ID NO: 50) | | (SEQ ID NO: 90) |
| Gdf8-460 | UACAAUAUAACAAAGUAG | Gdf8-1095 | GAUGUCCCCUAUAAACAUG |
| | U (SEQ ID NO: 51) | | (SEQ ID NO: 91) |
| Gdf8-465 | UAUAACAAAGUAGUAAAG | Gdf8-1111 | ACAUGCUGUAUUUCAAUGG |
| | G (SEQ ID NO: 52) | | (SEQ ID NO: 92) |
| Gdf8-468 | CAAAGUAGUAAAGGCACAA | Gdf8-1116 | UGGAAAAGAACAAAUAAUA |
| | (SEQ ID NO: 53) | | (SEQ ID NO: 93) |
| Gdf8-476 | AGUAGUAAAGGCACAAUU | Gdf8-1118 | AAGAACAAAUAAUAUAUGG |
| | A (SEQ ID NO: 54) | | (SEQ ID NO: 94) |
| Gdf8-484 | AGGCACAAUUAUGGAUAU | Gdf8-1121 | GAACAAAUAAUAUAUGGAA |
| | A (SEQ ID NO: 55) | | (SEQ ID NO: 95) |
| Gdf8-508 | UUAUGGAUAUACUUGAGG | Gdf8-1124 | CAAAUAAUAUAUGGAAAGA |
| | C (SEQ ID NO: 56) | | (SEQ ID NO: 96) |
| Gdf8-514 | GUCCAAAAACCUACAACGG | Gdf8-1128 | AUAAUAUAUGGAAAGAUAC |
| | (SEQ ID NO: 57) | | (SEQ ID NO: 97) |
| Gdf8-516 | AAACCUACAACGGUGUUUG | Gdf8-1141 | UAUAUGGAAAGAUACCAGC |
| | (SEQ ID NO: 58) | | (SEQ ID NO: 98) |
| Gdf8-524 | ACCUACAACGGUGUUUGUG | | |
| | (SEQ ID NO: 59) | | |

TABLE 5
Sequence of myostatin siRNAs optimized
using the Taxman algorithm.

| Name | 5′-3′ Sequnce |
|---|---|
| 152 | CCAGAAUAGAAGCCAUAAA (SEQ ID NO: 113) |
| 460 | GCACAAUUAUGGAUAUACU (SEQ ID NO: 114) |
| 548 | GUACAAGAUAUACUGGAAU (SEQ ID NO: 115) |
| 1039 | CCUAUAAACAUGCUGUAUU (SEQ ID NO: 116) |
| 938 | GCGAAUUUGUGUUUCUACA (SEQ ID NO: 117) |
| 612 | GAGUAUUGAUGUGAAGACA (SEQ ID NO: 118) |
| 149 | CCUCCAGAAUAGAAGCCAU (SEQ ID NO: 119) |
| 762 | GGUCAGAGUUACAGACACA (SEQ ID NO: 120) |
| 860 | CAGUGGAUUUCGAAGCUUU (SEQ ID NO: 121) |
| 500 | CAACGGUGUUUGUGCAGAU (SEQ ID NO: 122) |

Each ddRNAi plasmid was constructed so that the start of each shRNA sequence was at the +1 position of the native U6 snRNA transcripts. A XhoI restriction enzyme site was engineered downstream of the termination signal to allow screening for full-length shRNA products inserted into pGEM-T Easy. All final shRNA expression vectors consisted of the full length chicken U6 promoter, a shRNA sense sequence, a loop sequence, a shRNA antisense sequence, a termination sequence and a Xhol site. The loop sequence used in all shRNAs was 5′ 10 UUCAAGAGA 3′.

TABLE 6
Sequence and details of primers used.

| Name | Sequence 5′-3′ | Location/Feature |
|---|---|---|
| TD135 | CGAAGAACCGAGCGCTGC (SEQ ID NO: 99) | cU6-1 |
| TD148 | CTCGAGTTCCAAAAAAGCTGACCCTGAAGTTCAT | cU6-1 shEGFP |
| | CTCTCTTGAAGATGAACTTCAGGGTCAGCGAATA | |
| | TCTCTACCTCCTAGG (SEQ ID NO: 109) | |
| DS304 | CTCGAGTTCCAAAAAATTCCTGCAGTGGAGGAGC | cU6-1 shGdf8-258 |
| | TTCTCTTGAAAGCTCCTCCACTGCAGGAAGAATA | |
| | TCTCTACCTCCTAGG (SEQ ID NO: 110) | |
| DS305 | CTCGAGTTCCAAAAAATCCAGTCCCATCCAAAAG | cU6-1 shGdf8-913 |
| | CTCTCTTGAAGCTTTTGGATGGGACTGGAGAATA | |
| | TCTCTACCTCCTAGG (SEQ ID NO: 111) | |
| DS306 | CTCGAGTTCCAAAAAACAGGTGAGTGTGCGGGTA | cU6-1 shGdf8-1002 |
| | TTCTCTTGAAATACCCGCACACTCACCTGGAATAT | |
| | CTCTACCTCCTAGG (SEQ ID NO: 112) | |

### Testing Selected shRNAs for Knockdown of Gdf8 Gene Expression

A reporter gene expression assay was used to test the three selected shRNAs for silencing of Gdf8. The reporter gene was a transcriptional gene fusion of Gdf8 inserted downstream of the 3′ end of the Enhanced Green Fluorescent Protein (EGFP) gene in pEGFP-C (Clontech). The reporter plasmid was constructed as follows: cDNA of Gdf8 was reverse transcribed from total RNA isolated from 7 day old embryo's and cloned into the multiple cloning site of pGEM-T Easy (Promega). The Gdf8 insert was removed from the cloning vector as a NotI fragment and cloned downstream of the EGFP gene in pEGFP-C (Clontech). The resulting plasmid was named pEGFP-Gdf8. This plasmid was transfected into chicken DF-1 cells and expression of the transcriptional gene fusion was confirmed by measuring EGFP fluorescence using flow cytometry as described below.

Gdf8 gene silencing assays were conducted by co-transfecting DF-1 cells with the pEGFP-Gdf8 reporter plasmid and each of the ddRNAi plasmids expressing the Gdf8 specific or EGFP control shRNAs. The co-transfection experiments were performed as follows: DF-1 cells (ATCC CRL-12203, chicken fibroblast) were maintained in a humidified atmosphere containing 5% CO₂ at 37° C. in Dulbecco's Modified Eagle's Medium (DMEM) containing 4.5 g/l glucose, 1.5 g/l sodium bicarbonate, 10% foetal calf serum (FCS), 2 mM L-glutamine supplemented with penicillin (100 U/ml) and streptomycin (100 μg/ml). DF1 cells were passaged as required using 0.25% (w/v) trypsin-ethylenediaminetetraacetic acid (EDTA).

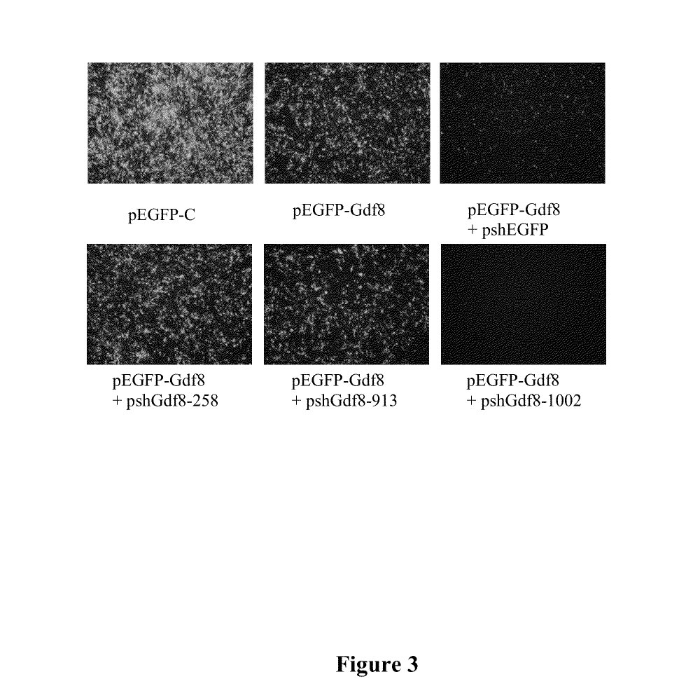

Co-transfection of pEGFP-Gdf8 and ddRNAi plasmids for EGFP-Gdf8 fusion silencing assays was conducted in DF-1 cells grown to 80-90% confluence, in 8 well chamber slides (Nunc) for fluorescence microscopy analysis. Cells were transfected with a total of 1 μg of plasmid DNA, per well, using Lipofectamine™2000 transfection reagent (Invitrogen). EGFP expression was analysed in transfected DF-1 cells at 60 hours post-transfection as follows: Co-transfected cells in 8-well chamber slides were washed with PBSA, chamber slide housings were removed and coverslips mounted over cell monolayers. Microscopy was performed using a Leica DM LB Fluorescence Microscope (Leica Microsystems, Germany) and images were captured at 50× magnification using a Leica DC300F colour digital camera (Leica Microsystems, Germany) and Photoshop 7.0 imaging software (Adobe®). The results are shown in FIG. 3. shGdf8-1002 was very effectively silenced expression of the fusion transcript and would therefore be an excellent candidate for silencing of the native Gdf8 transcript.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive. The present application claims priority from U.S. 60/943,708 filed 13 Jun. 2007, the entire contents of which are incorporated herein in their entirety by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Amarzguioui et al. (2004) Biochem Biophys Res Commun 316:1050-1058
Elbashire et al. (2001) Nature 411:494-498
Hori et al. (2000) Mol Biol Cell 11:3645-3660
Freier et al. (1986) Proc Natl Acad Sci USA 83:9373-9377
Needleman and Wunsch (1970) J Mol Biol 48: 443-453
O'Neill et al. (2000) Dev Genes Evol 210:243-249
Raymond et al. (1999) Dev Biol. 215:208-220
Reynolds et al. (2004) Nat. Biotech., 22:326-330
Smith et al. (1999) Nature 402:601-602
Smith et al. (2000) Nature 407: 319-320.
Taxman et al. (2006) BMC Biotechnol, Jan 24, 6:7
Waterhouse et al. (1998) Proc Natl Acad Sci USA 95:13959-13964

We claim:

1. A method of modifying a trait of an avian, the method comprising administering to an avian egg at least one RNA molecule comprising a double-stranded region (dsRNA), wherein the RNA molecule results in a reduction in the level of at least one RNA molecule and/or protein in the egg, and wherein
i) the method does not comprise electroporating the egg, and/or
ii) the RNA molecule is administered to the air sac, yolk sac or chorion allantoic fluid.

2. The method of claim 1, wherein the dsRNA is a siRNA or a shRNA.

3. The method of claim 1, wherein the trait is a production trait.

4. The method of claim 3, wherein the production trait is muscle mass or sex.

5. The method of claim 4, wherein the production trait is sex and the nucleic acid molecule reduces the level of a protein encoded by a DMRT1 gene.

6. The method of claim 5, wherein the nucleic acid molecule comprises at least one nucleotide sequence selected from:
(SEQ ID NO: 11)
CCAGUUGUCAAGAAGAGCA
(SEQ ID NO: 12)
GGAUGCUCAUUCAGGACAU
(SEQ ID NO: 13)
CCCUGUAUCCUUACUAUAA
(SEQ ID NO: 14)
GCCACUGAGUCUUCCUCAA
(SEQ ID NO: 15)
CCAGCAACAUACAUGUCAA
(SEQ ID NO: 16)
CCUGCGUCACACAGAUACU
(SEQ ID NO: 17)
GGAGUAGUUGUACAGGUUG
(SEQ ID NO: 18)
GACUGGCUUGACAUGUAUG
(SEQ ID NO: 19)
AUGGCGGUUCUCCAUCCCU,
or a variant of any one thereof.

7. The method of claim 1, wherein the nucleic acid molecule is administered by injection.

8. The method of claim 1, wherein the avian is selected from chickens, ducks, turkeys, geese, bantams and quails.

9. An avian produced using the method of claim 1.

10. An isolated and/or exogenous nucleic acid molecule comprising a double-stranded region which reduces the level of at least one RNA molecule and/or protein when administered to an avian egg.

11. The nucleic acid molecule of claim 10 which is a dsRNA molecule.

12. The nucleic acid molecule of claim 10 which reduces the level of a protein encoded by a DMRT1 gene or a myostatin gene.

13. The nucleic acid molecule of claim 12 which comprises at least one nucleotide sequence selected from:
(SEQ ID NO: 11)
CCAGUUGUCAAGAAGAGCA
(SEQ ID NO: 12)
GGAUGCUCAUUCAGGACAU
(SEQ ID NO: 13)
CCCUGUAUCCUUACUAUAA
(SEQ ID NO: 14)
GCCACUGAGUCUUCCUCAA
(SEQ ID NO: 15)
CCAGCAACAUACAUGUCAA
(SEQ ID NO: 16)
CCUGCGUCACACAGAUACU
(SEQ ID NO: 17)
GGAGUAGUUGUACAGGUUG
(SEQ ID NO: 18)
GACUGGCUUGACAUGUAUG
(SEQ ID NO: 19)
AUGGCGGUUCUCCAUCCCU
(SEQ ID NO: 20)
AAGCUAGCAGUCUAUGUUU
(SEQ ID NO: 21)
GCUAGCAGUCUAUGUUUAU
(SEQ ID NO: 22)
CGCUGAAAAAGACGGACUG
(SEQ ID NO: 23)
AAAGACGGACUGUGCAAUG
(SEQ ID NO: 24)
AGACGGACUGUGCAAUGCU
(SEQ ID NO: 25)
UGCUUGUACGUGGAGACAG
(SEQ ID NO: 26)
UACAAAAUCCUCCAGAAUA
(SEQ ID NO: 27)
AAUCCUCCAGAAUAGAAGC
(SEQ ID NO: 28)
UCCUCCAGAAUAGAAGCCA
(SEQ ID NO: 29)
UAGAAGCCAUAAAAAUUCA
(SEQ ID NO: 30)
GCCAUAAAAAUUCAAAUCC
(SEQ ID NO: 31)
AAAUUCAAAUCCUCAGCAA
(SEQ ID NO: 32)
AUUCAAAUCCUCAGCAAAC
(SEQ ID NO: 33)
AUCCUCAGCAAACUGCGCC
(SEQ ID NO: 34)
ACUGCGCCUGGAACAAGCA
(SEQ ID NO: 35)
CAAGCACCUAACAUUAGCA
(SEQ ID NO: 36)
GCACCUAACAUUAGCAGGG
(SEQ ID NO: 37)
CAUUAGCAGGGACGUUAUU
(SEQ ID NO: 38)
GCAGCUUUUACCCAAAGCU
(SEQ ID NO: 39)
UUCCUGCAGUGGAGGAGCU
(SEQ ID NO: 40)
CUGAUUGAUCAGUAUGAUG
(SEQ ID NO: 41)
GACGAUGACUAUCAUGCCA
(SEQ ID NO: 42)
CCGAGACGAUUAUCACAAU
(SEQ ID NO: 43)
UGCCUACGGAGUCUGAUUU
(SEQ ID NO: 44)
AUGGAGGGAAAACCAAAAU
(SEQ ID NO: 45)
AACCAAAAUGUUGCUUCUU
(SEQ ID NO: 46)
CCAAAAUGUUGCUUCUUUA
(SEQ ID NO: 47)
AAUGUUGCUUCUUUAAGUU
(SEQ ID NO: 48)
UGUUGCUUCUUUAAGUUUA
(SEQ ID NO: 49)
GUUUAGCUCUAAAAUACAA
(SEQ ID NO: 50)
AAUACAAUAUAACAAAGUA
(SEQ ID NO: 51)
UACAAUAUAACAAAGUAGU
(SEQ ID NO: 52)
UAUAACAAAGUAGUAAAGG
(SEQ ID NO: 53)
CAAAGUAGUAAAGGCACAA
(SEQ ID NO: 54)
AGUAGUAAAGGCACAAUUA
(SEQ ID NO: 55)
AGGCACAAUUAUGGAUAUA
(SEQ ID NO: 56)
UUAUGGAUAUACUUGAGGC
(SEQ ID NO: 57)
GUCCAAAAACCUACAACGG
(SEQ ID NO: 58)
AAACCUACAACGGUGUUUG
(SEQ ID NO: 59)
ACCUACAACGGUGUUUGUG
(SEQ ID NO: 60)
CGGUGUUUGUGCAGAUCCU
(SEQ ID NO: 61)
GCCCAUGAAAGACGGUACA
(SEQ ID NO: 62)
AGACGGUACAAGAUAUACU
(SEQ ID NO: 63)
GAUAUACUGGAAUUCGAUC
(SEQ ID NO: 64)
UUCGAUCUUUGAAACUUGA
(SEQ ID NO: 65)
ACUUGACAUGAACCCAGGC
(SEQ ID NO: 66)
CCCAGGCACUGGUAUCUGG
(SEQ ID NO: 67)
GACAGUGCUGCAAAAUUGG
(SEQ ID NO: 68)
AAUUGGCUCAAACAGCCUG
(SEQ ID NO: 69)
UUGGCUCAAACAGCCUGAA
(SEQ ID NO: 70)
ACAGCCUGAAUCCAAUUUA
(SEQ ID NO: 71)
UCCAAUUUAGGCAUCGAAA
(SEQ ID NO: 72)
UUUAGGCAUCGAAAUAAAA
(SEQ ID NO: 73)
AUAAAAGCUUUUGAUGAGA
(SEQ ID NO: 74)
AAGCUUUUGAUGAGACUGG
(SEQ ID NO: 75)
GCUUUUGAUGAGACUGGAC
(SEQ ID NO: 76)
GAUGGAUUGAACCCAUUUU
(SEQ ID NO: 77)
CCCAUUUUUAGAGGUCAGA
(SEQ ID NO: 78)
ACGGUCCCGCAGAGAUUUU
(SEQ ID NO: 79)
CGGAAUCCCGAUGUUGUCG
(SEQ ID NO: 80)
UCCAGUCCCAUCCAAAAGC
(SEQ ID NO: 81)
GCUUUUGGAUGGGACUGGA
(SEQ ID NO: 82)
AAGAUACAAAGCCAAUUAC
(SEQ ID NO: 83)
GAUACAAAGCCAAUUACUG
(SEQ ID NO: 84)
AGCCAAUUACUGCUCCGGA
(SEQ ID NO: 85)
UUACUGCUCCGGAGAAUGC
(SEQ ID NO: 86)
UGCGAAUUUGUGUUUCUAC
(SEQ ID NO: 87)
CAGGUGAGUGUGCGGGUAU
(SEQ ID NO: 88)
AUACCCGCACACUCACCUG
(SEQ ID NO: 89)
GCAAAUCCCAGAGGUCCAG
(SEQ ID NO: 90)
AUCCCAGAGGUCCAGCAGG
(SEQ ID NO: 91)
GAUGUCCCCUAUAAACAUG
(SEQ ID NO: 92)
ACAUGCUGUAUUUCAAUGG
(SEQ ID NO: 93)
UGGAAAAGAACAAAUAAUA
(SEQ ID NO: 94)
AAGAACAAAUAAUAUAUGG
(SEQ ID NO: 95)
GAACAAAUAAUAUAUGGAA
(SEQ ID NO: 96)
CAAAUAAUAUAUGGAAAGA
(SEQ ID NO: 97)
AUAAUAUAUGGAAAGAUAC
(SEQ ID NO: 98)
UAUAUGGAAAGAUACCAGC
(SEQ ID NO: 113)
CCAGAAUAGAAGCCAUAAA
(SEQ ID NO: 114)
GCACAAUUAUGGAUAUACU
(SEQ ID NO: 115)
GUACAAGAUAUACUGGAAU
(SEQ ID NO: 116)
CCUAUAAACAUGCUGUAUU
(SEQ ID NO: 117)
GCGAAUUUGUGUUUCUACA
(SEQ ID NO: 118)
GAGUAUUGAUGUGAAGACA
(SEQ ID NO: 119)
CCUCCAGAAUAGAAGCCAU
(SEQ ID NO: 120)
GGUCAGAGUUACAGACACA
(SEQ ID NO: 121)
CAGUGGAUUUCGAAGCUUU
(SEQ ID NO: 122)
CAACGGUGUUUGUGCAGAU,
or a variant of any one thereof.

14. A vector encoding a nucleic acid molecule, or a single strand thereof, of claim 10.

15. A host cell comprising an exogenous nucleic acid molecule, or a single strand thereof, of claim 10.

16. A composition comprising a nucleic acid molecule, or a single strand thereof, of claim 10.

17. An avian egg comprising a nucleic acid molecule, or a single strand thereof, of claim 10.

18. A kit comprising a nucleic acid molecule, or a single strand thereof, of claim 10.
